(19) United States
(12) Patent Application Publication
(10) Pub. No.: US 2002/0172936 A1
(43) Pub. Date: Nov. 21, 2002

(54) APPARATUS AND METHOD FOR DISEASE DETECTION

(76) Inventors: JOSEPH M. CANTER, LEXINGTON, MA (US); YONGWU YANG, BELMONT, MA (US); WANGLONG ZHOU, READING, MA (US); VICTOR S. SAPIRSTEIN, POUND RIDGE, NY (US); MELVIN P. EHRLICH, ROSLYN ESTATES, NY (US); JAMES S. HARRISON, RINGWOOD, NJ (US); EUGENE KATSMAN, ARLINGTON, MA (US); OMANAND KOUL, BURLINGTON, MA (US); MICHAEL Y. LU, LEXINGTON, MA (US); MICHAEL A. GREENWALD, BROOKLINE, MA (US)

Correspondence Address: PENNIE & EDMONDS LLP, 1667 K STREET NW, SUITE 1000, WASHINGTON, DC, 20006

(21) Appl. No.: 09/436,207

(22) Filed: Nov. 8, 1999

Related U.S. Application Data
(63) Continuation-in-part of application No. 09/224,141, filed on Dec. 31, 1998, now Pat. No. 6,265,151.
(60) Provisional application No. 60/079,556, filed on Mar. 27, 1998.

(51) Int. Cl. C12Q 1/70

(52) U.S. Cl. 435/5

(57) ABSTRACT

The present invention relates to an apparatus and method for disease detection. The apparatus and method use a radiation source to irradiate a sample. An analyzer measures the absorption and/or emission spectra from the sample to detect the presence of the disease state in the sample. In this regard, a comparator may compare the measured spectra with the spectrum of a control. Analysis of the parameters of the spectra including, but not limited to, peak intensity wavelength, amplitude at the peak intensity, area ratio of left and right portions of the emission spectra, and shifts of the peak intensity wavelength, allows determination of HIV infection, Hepatitis A, B and C, and other diseases. Selective absorbents, such as C-M Affi Gel Blue and activated charcoal, may be used to treat the samples before measurements, which is found to improve discrimination of diseased and non-diseased samples. The present invention is capable of detecting HIV infection at a stage when it is still undetectable by conventional diagnosis methods.

### CROSS REFERENCE TO RELATED APPLICATIONS

This application is a continuation-in-part of U.S. application Ser. No. 09/224,141, filed Dec. 31, 1998, which claims the benefit of U.S. Provisional Application No. 60/079,556, filed on Mar. 27, 1998 under 35 U.S.C. § 119(e).

### FIELD OF THE INVENTION

The present invention relates to the detection of the presence of disease and more particularly to detection of the presence of HIV infection, Hepatitis A, B and C infection, and other diseases by optical means.

### BACKGROUND OF THE INVENTION

Optical systems have been used to determine chemical compositions of matters. Such optical systems include lasers or other radiation sources which have long provided impetus to a wide range of spectroscopic investigations. Due to practical considerations, lasers are the preferred radiation source for many of these investigations. The advantages of lasers, such as their monochromaticity (ability to operate within a very narrow wavelength range), very high intensity compared to incoherent light sources, such as mercury lamps, and availability of inexpensive laser sources, are allowing many types of experiments and measurements not previously possible. Nowadays, the use of lasers in experiments or tests, such as absorbance or fluorescence measurements in chemical analysis, is routine.

Experiments or measurements based on the absorption or emission of radiation upon illumination of a sample by a radiation source have long been used to determine the nature of a sample's various components. The principle is based on the fact that given a high enough resolution for the measuring spectroscopic apparatus, every chemical component will give rise to a different absorption or emission (referred to hereinafter as fluorescence) spectrum which is a plot of the intensity of radiation absorbed or emitted by a sample measured at various wavelengths of the incident or emitted radiation. The absorption or fluorescence spectrum therefore corresponds to a fingerprint of a given chemical component, allowing its discrimination from other components present in the same sample.

Absorption or fluorescence spectroscopy has been an important technique for identification of unknown biological substances in aqueous (water-based) solutions. An absorption or fluorescence spectrum, of an aqueous solution, however, may be complicated by the fact that absorption bands of the various biological substances in the solution sometimes overlap, particularly in the case of large molecules, such as biomolecules, making assignments of their origins difficult. There is, in addition to absorption or fluorescence signals originating from a component of interest, a large amount of background noise generated by water and other constituents present in biological samples, such as blood or plasma.

Currently, a number of simple-to-operate, non-optical based analyzers for detection of certain infectious diseases, such as HIV infection, are commercially available. Examples of such analyzers are Ektachem DT 60, Ektachem 700P, Reflotron, and Seralyzer. These systems allow tests to be performed in settings outside traditional laboratories, e.g., outpatient clinics, physicians' offices, and even shopping malls, schools, or churches. In addition, these systems offer the advantage of being economical, compact, lightweight, easy-to-operate, convenient, and requiring only a small amount of test sample. Some of these analyzers also have the potential for providing test accuracy precision similar to those obtained from more sophisticated analyzers used in large clinical laboratories. However, none of these commercially available analyzers are understood to be optical-based. In addition, no such simple-to-operate system exists for detecting Hepatitis A, B or C infection.

Another drawback of available analyzers for detecting HIV infection is that they are not sufficiently sensitive and thus incapable of detecting HIV infection at the early stage of the infection. Only when the infection has occurred for some time such that the antibody or antigens of the HIV viruses in the blood sample reach detectable level, can the HIV infection be detected by conventional analyzers.

U.S. Pat. No. 5,267,152 to Yang et al. discloses an optical technique for a non-invasive measurement of blood glucose concentration using a near-infrared photodiode laser. Yang et al. determines the blood glucose concentration using an algorithm based upon the characteristic translational, vibrational and rotational motions of the molecules in the blood resulting from the excitation of the sample with a diode laser, and then comparing the optical signal from light reflected off the blood constituents with a calibration curve previously stored in the memory of a microprocessor. However, Yang et al. does not measure changes in the emission spectra of biological samples as a result of changes in the amounts of certain metabolites in the samples.

U.S. Pat. No. 5,258,788 to Furuya discloses an optical method for measuring the protein composition and concentration of the aqueous humor of the eye which, in addition to proteins, also contains blood cells. However, Furuya does not measure the emission of light by the constituents of a sample as a result of irradiation of the sample with a laser beam. Instead, it measures the scattering of incident light off the sample molecules rather than on the emission of light by the molecules themselves as a result of irradiation with a laser beam.

U.S. Pat. No. 5,238,810 and U.S. Pat. No. 5,252,493 both to Fujiwara et al. disclose an optical-based immunoassay method in which antigens or antibodies are labeled with micro-particles of a magnetic substance to form a magnetic labeled body, thus allowing determination of minute amounts of the antigen or antibody. It would be desirable if viruses be optically detected without the use of magnetic particles.

It is therefore an object of the present invention to provide identification of a wavelength range of an excitation laser beam within which the fluorescence spectrum of the sample will provide information that can be used to detect and identify any infectious disease present in the sample. Also the present invention provides for identification of a fluorescence wavelength range within which the fluorescence spectrum of a sample will yield information that may be used to detect and identify any infectious organism contained in the sample. Also various parameters obtained from the fluorescent spectrum of a sample may be used to detect and identify any infectious disease contained in the sample. Thus the present invention provides apparatus and method for detecting HIV infection at an earlier stage when they are yet to be detectable by conventional methods.

Thus, there exists a need for an improved apparatus and method for disease detection.

### SUMMARY OF THE INVENTION

The method for detecting the presence of a diseased state (or infection) according to the present invention includes the steps of obtaining a specimen, preparing a sample from the specimen, irradiating the sample, obtaining absorption and/or emission spectra from the irradiated sample, and analyzing the spectra to determine the presence of the diseased state (or infection). The sample can be any biological material, such as plasma, serum, blood particles, or blood. The analysis of the spectra can be determining variation of the spectra from a spectrum characteristic of a non-infected state to detect the presence of the infection.

The apparatus for detecting the presence of a diseased state in a sample according to the present invention includes a radiation source, such as a laser or a filtered light source, and an analyzer operatively associated with the sample for measuring at least one of the absorption and emission spectra from the sample to detect the presence of the diseased state in the sample. The sample can be held in a sample holder disposed relative to the radiation source so that at least a portion of the sample can be irradiated. The sample holder may be a flow-through cell or other suitable container.

The analyzer can include a spectrometer, such as a CCD spectrometer or a time-resolved spectrometer, and a comparator to compare the at least one absorption and emission spectra with a signal characteristic of a non-diseased state. The analyzer can also include an adjustable polarizing filter arranged to measure spectra at two orthogonal polarizations. In an exemplary embodiment, a memory unit is coupled with the comparator and contains a plurality of pre-determined signals characteristic of non-diseased states.

In a preferred embodiment, an excitation at a selected wavelength is irradiated onto a sample, and the resultant fluorescence spectra between a particular wavelength range of interest is detected and analyzed. The present invention allows for the differentiation between a non-diseased sample and a diseased sample.

In accordance with the preferred embodiment of the present invention, an excitation wavelength of about 355 nanometers (nm) is selected to generate fluorescence from a sample that yields useful information for disease detection and identification. In addition, a fluorescence wavelength range of about 380 nm to 600 nm is selected for fluorescence detection, because within this wavelength range, the fluorescence spectrum of the samples is found to provide useful information for disease detection and identification.

With respect to specific diseases, several parameters of these spectra have been identified which clearly differentiate HIV positive plasma from HIV negative plasma, or plasma infected with Hepatitis A, B or C from normal uninfected plasma. These parameters includes, but not limited to, the wavelength at which the peak emission intensity is obtained; the amplitude of the peak emission; and the area ratio of the left and right portions of the emission spectra. In addition, HIV positive and negative plasmas may be distinguished by using an algorithm having one or more of these parameters as variables.

In the present invention, the samples being analyzed may be first treated with selective absorbents, such as C-M Affi Gel Blue or activated charcoal, to improve the discrimination of these parameters for diseased and non-diseased states. For example, it is found that C-M Affi Gel Blue significantly shifts the peak wavelength of an emission toward a lower wavelength. Advantageously, this peak wavelength shift is significantly greater for HIV positive samples than for HIV negative (normal) samples, thus allowing improved discrimination of the HIV positive and negative samples on the basis of the peak wavelengths of the samples.

In addition, for samples treated with C-M Affi Gel Blue, it is also found that the intensity of the fluorescence peak is reduced more in HIV negative samples than in HIV positive samples. This improves the discrimination of the HIV positive samples from HIV negative samples on the basis of intensity or amplitude of the emission peak. Further, it is found that HIV positive samples treated with activated charcoals exhibit a smaller shift of the emission band than in HIV negative samples treated with activated charcoal, which enhances the discrimination of the infected and uninfected samples on the basis of peak wavelengths.

The present invention provides a more sensitive detection of HIV infection and other diseases, particularly at the early stage of the infection than conventional diagnosis methods. For example, in instances where samples from an individual infected with HIV viruses at the early stage when the infection is still undetectable by conventional diagnosis, it is detected by using the method and apparatus of the present invention.

### BRIEF DESCRIPTION OF THE DRAWINGS

These and other features, objects, and advantages of the present invention will become more apparent from the following detailed description in conjunction with the appended drawings in which:

FIG. 1 is a schematic block diagram of a disease detection system of the present invention;

FIG. 2A is a schematic block diagram of an exemplary embodiment of the detection system according to the present invention;

FIG. 2B is a top view of the system of FIG. 2A;

FIG. 3 shows fluorescence spectra of an HIV negative plasma sample and HIV positive plasma sample produced at an excitation wavelength of 355 nm and detected within the wavelength range of 350 nm to 600 nm;

FIG. 3A shows fluorescence spectra of a group of HIV positive plasma samples and a group pf HIV negative plasma samples;

FIG. 4 illustrates the fluorescence spectra of HIV negative and positive samples as well as such samples treated with DEAE-Affi Gel Blue;

FIG. 5 illustrates the fluorescence spectra of peptide, HIV positive and negative samples;

FIG. 6A illustrates the fluorescence spectra of holo-albumin (HSA) and fatty acid free albumin;

FIG. 6B illustrates the fluorescence spectrum of LDL;

FIG. 7 illustrates the fluorescence spectra of HIV positive and negative samples as well as such samples treated with TCA;

FIG. 8 illustrates the fluorescence spectra of HIV positive and negative samples as well as such samples acidified and dialyzed;

FIG. 9A illustrates the fluorescence spectra of a group of HIV positive and HIV negative samples and an HSA sample;

FIG. 9B illustrates the fluorescence spectrum of LDL;

FIG. 10 illustrates the fluorescence spectra of HIV positive and HIV negative samples, respectively, and such samples treated with CM-Affi Gel Blue (CM-AGB);

FIG. 11 illustrates the fluorescence spectra of HIV positive and HIV negative samples, an NADH sample and a sample of a mixture of NADH and HSA;

FIG. 12 illustrates the fluorescence spectra of an NADH sample and such sample treated with activated charcoal;

FIG. 13 illustrates the fluorescence spectra of thiocrome, a mixture of thiocrome, NADH and HSA, and HIV positive and negative samples;

FIGS. 14A-D illustrate the fluorescence spectra of NADH, thiocrome, riboflavin, and HDL;

FIG. 15 illustrates the fluorescence spectrum of an HIV negative sample and a reconstructed spectrum;

FIG. 16 illustrates the fluorescence spectrum of an HIV positive sample and a reconstructed spectrum;

FIG. 16A illustrates the fluorescence spectra of a group of HIV positive samples and a group of HIV negative samples;

FIG. 16B illustrates the fluorescence spectra of the HIV positive and HIV negative samples of FIG. 16A but normalized at 550 nm;

FIG. 16C illustrates the fluorescence spectra of the HIV positive and HIV negative samples of FIG. 16A treated with CM-AGB;

FIG. 16D illustrates the fluorescence spectra of the CM-AGB treated HIV positive and HIV negative samples of FIG. 16C but normalized at 550 nm;

FIG. 17A illustrates the fluorescence spectra of another group of HIV positive samples and another group of HIV negative samples;

FIG. 17B illustrates the fluorescence spectra of the HIV positive and HIV negative samples of FIG. 17A but normalized at 550 nm;

FIG. 17C illustrates the fluorescence spectra of the HIV positive and HIV negative samples of FIG. 17A treated with CM-AGB;

FIG. 17D is the fluorescence spectra of the CM-AGB treated HIV positive and CM-AGB treated HIV negative samples of FIG. 17C but normalized at 550 nm;

FIG. 18 illustrates the fluorescence spectra of an HIV positive plasma and an HIV negative plasma samples for demonstrating the definition of an “area ratio” parameter;

FIG. 19 shows emission peak amplitudes of a group of HIV positive and negative samples normalized at 550 nm with and without CM-AGB treatment;

FIG. 20 shows the emission peak wavelengths of the same HIV positive and negative samples of FIG. 19 with and without CM-AGB treatment;

FIG. 21 shows the area ratios of the same HIV positive and negative samples of FIG. 19 with and without CM-AGB treatment;

FIG. 22 shows amplitude changes of the same HIV positive and negative samples of FIG. 19 after the CM-AGB treatment;

FIG. 23 shows wavelength changes of the same HIV positive and negative samples of FIG. 19 after the CM-AGB treatment;

FIG. 24 shows area ratio changes of the HIV positive and negative samples after the CM-AGB treatment;

FIG. 25 illustrates a process flow chart according to the present invention;

FIGS. 26A-C show early detection of HIV infection of untreated samples as compared with control samples;

FIGS. 27A-C show early detection of HIV infection using the same samples of FIGS. 26A-C after CM-AGB treatment as compared with control samples;

FIG. 28 illustrates a table setting forth time and test results of the same samples of FIG. 26A-C tested by conventional methods;

FIG. 29 shows the emission peak amplitude differences of Hepatitis infected samples between the samples treated with CM-AGB and the same samples treated with activated charcoal and their comparison to those of non-Hepatitis infected control samples;

FIG. 30 shows the area ratio differences of the same Hepatitis infected samples of FIG. 29 between the samples treated with CM-AGB and the same samples treated with activated charcoal and their comparison to those of non-Hepatitis infected control samples;

FIG. 31 shows the peak emission wavelengths of the same Hepatitis infected samples of FIG. 29 as compared with those of non-Hepatitis infected control samples;

FIG. 32 shows peak emission amplitude differences of the same samples of FIG. 29 between the samples treated with CM-AGB and the same samples treated with activated charcoal and their comparison to those of non-Hepatitis infected control samples;

FIG. 33 shows peak emission wavelength differences of the same samples of FIG. 29 between the samples treated with CM-AGB and the same samples treated with activated charcoal and their comparisons to that of non-Hepatitis infected control samples;

FIG. 34 shows a comparison of the parameters of peak emission amplitude of samples treated with CM-AGB divided by peak emission wavelength of the same sample but treated with activated charcoal between certain Hepatitis infected samples and nonepatitis infected control samples;

FIG. 35 shows a comparison of the parameters of certain samples for Hepatitis detection;

FIG. 36 shows area ratios of certain samples for Hepatitis detection;

FIGS. 37 shows normalized peak amplitude measurement results for Hepatitis detection;

FIG. 38 shows peak wavelength measurement results for Hepatitis detection;

FIG. 39 shows area ratio changes of certain treated samples for Hepatitis detection;

FIG. 40 shows amplitude changes of certain treated samples for Hepatitis detection;

FIG. 41 shows peak wavelength changes of certain treated samples for Hepatitis detection;

FIG. 42 shows area ratio changes of certain treated samples for Hepatitis detection;

FIG. 43 shows amplitude changes of certain treated samples for Hepatitis detection;

FIG. 44 shows peak wavelength changes of certain treated samples for Hepatitis detection;

FIG. 45 shows composite parameters of certain samples for Hepatitis detection; and

FIG. 46 shows different composite parameters of certain samples for Hepatitis detection.

### DETAILED DESCRIPTION OF THE INVENTION

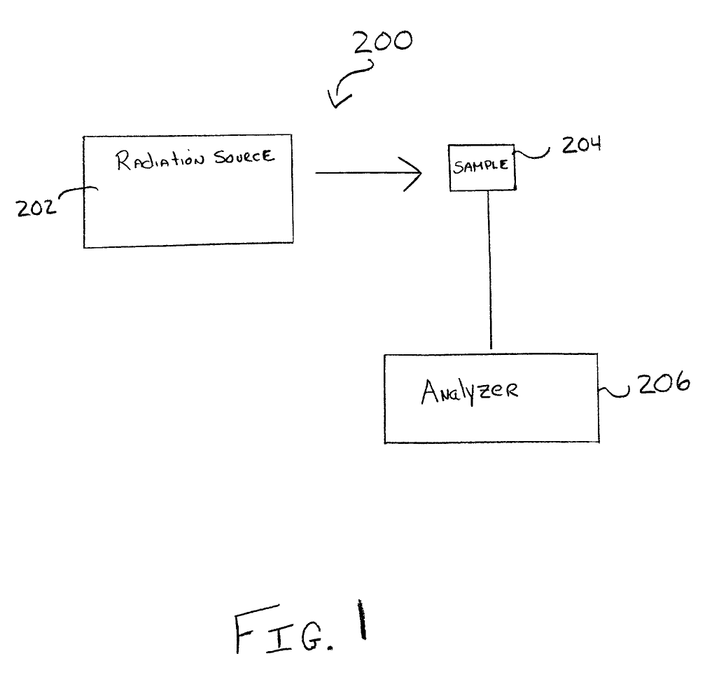

FIG. 1 schematically shows a detection system 200 according to the present invention. Detection system 200 is useful for detecting the presence of a diseased state, such as an infection, in a sample and utilizes the bundling of a radiation or light input (L), biologic phenomena (B) and signal recognition (S), which is referred hereinafter as an LBS system. Detection system 200 includes a radiation source 202 for irradiating a specimen or sample 204. As used throughout this application, the term “specimen” refers to a biological material that is analyzed substantially without processing, and the term “sample” refers to the product that results from processing of a specimen, e.g., plasma is the sample that results after removing the cellular material from a blood specimen. Unless otherwise indicated, these terms are used interchangeably throughout this application. Sample 204 can be in any physical form, i.e., solid, liquid, or gas. Radiation source 202 can be any suitable source that emits radiation of the desired wavelength(s), such as a laser, xenon flash lamp, deuterium lamp, or other light source filtered through an appropriate bandpass filter.

Radiation source 202 emits radiation in a wavelength range selected to cause sample 204 to fluoresce and/or absorb at least a portion of the radiation to yield useful information that can be used for disease detection and identification. In this regard, detection system 200 includes an analyzer 206 operatively associated with sample 204 for measuring the absorption and/or emission spectra from sample 204 to detect the presence of the diseased state in sample 204. In a preferred embodiment, analyzer 206 includes a spectrometer for recording the spectra from sample 204 and a comparator for comparing the absorption and/or emission spectra with a signal characteristic of a non-diseased state.

The use of detection system 200 for determining the presence of infectious disease in human plasma will now be described. Excitation of human plasma by radiation source 202 having a wavelength within the range of about 270 nm to 400 mn, and more preferably within the range of about 310 mn to 370 mn, such as 355 nm, elicits an emission spectrum which is attributed to a set of relatively lipophilic molecules associated with the metabolic status of the individual, such relatively lipophilic molecules being in large part bound to albumin and to low and very low density lipoproteins. Applicants found that if the excitation wavelength is less than 270 nm or greater than 400 mn, the background fluorescence signal from human plasma that is not believed to significantly contribute to infectious disease detection becomes so great that signals useful for infectious disease detection are not easily distinguishable from this background signal. In addition, according to the present invention, a fluorescence wavelength range of about 380 nm to about 600 mn is identified by Applicants as the fluorescence wavelength range that provides useful information for disease detection and identification. As will be detailed below, several parameters of these spectra have been identified which clearly differentiate HIV positive plasmas from HIV negative plasmas; or plasmas infected with Hepatitis A, B or C and normal, uninfected plasmas. According to the present invention, HIV positive and negative plasmas are distinguished by their respective wavelengths at which their respective emission intensity profile exhibit maximum emission intensities (λₘₐₓ), and by the relative values of such maximum intensities (Iₘₐₓ). In addition, according to the present invention, additional parameters, beside λₘₐₓ and I_{max,} for distinguishing HIV positive plasmas from HIV negative plasmas are also provided. One of such parameters is the so-called area ratio, Aᵣ, which is defined in detail below.

In accordance with the present invention, chromatography of the plasma through certain absorbents differentially alter these parameters in HIV positive plasma and HIV negative plasma samples, allowing a greater identification and distinction of an HIV positive sample. These absorbents include C-M Affi Gel Blue (hereinafter also referred to as “CM-AGB”) and activated charcoal. It is found that the emission spectra of a plasma sample, after it is treated with CM-AGB, exhibit a significant shift in wavelength towards lower wavelength. Advantageously, this shift is significantly greater for HIV positive samples (about 12-18 nm) than for HIV negative (normal) samples (about 4-8 nm). This effect helps to more clearly discriminate the HIV positive samples and HIV negative samples. It is also found that the intensity of the fluorescence peak, Iₘₐₓ, is reduced more in HIV negative plasma samples than in HIV positive plasma samples by CM-AGB treatment.

Applicants also found that activated charcoal treatment of the plasma samples results, advantageously, in a relatively smaller shift (towards shorter wavelengths) of the emission band for HIV positive samples (about 1-2 nm) than for HIV negative samples (about 5-8 nm).

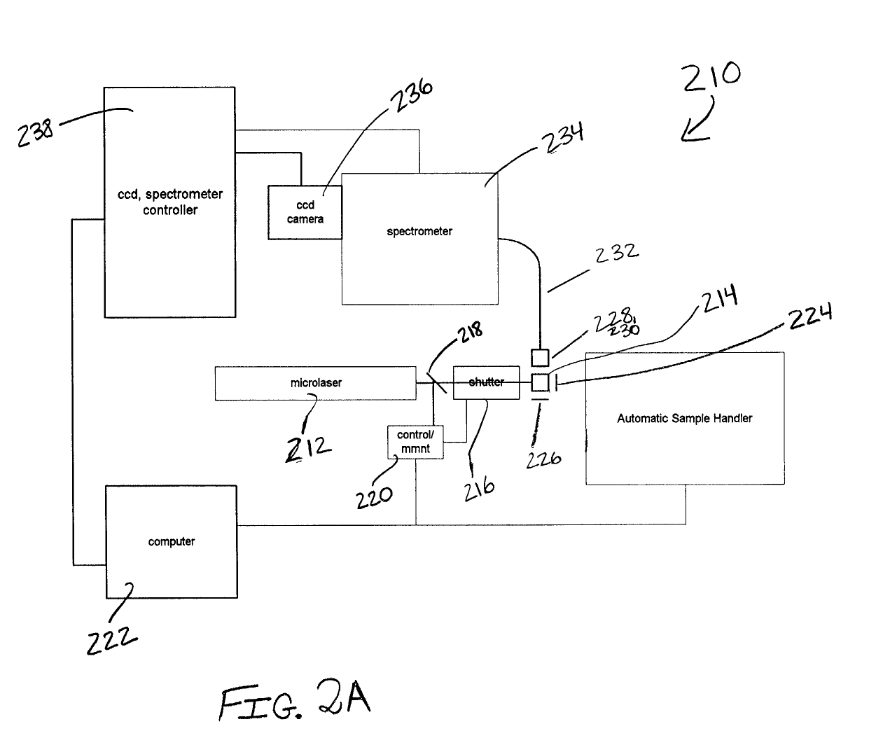

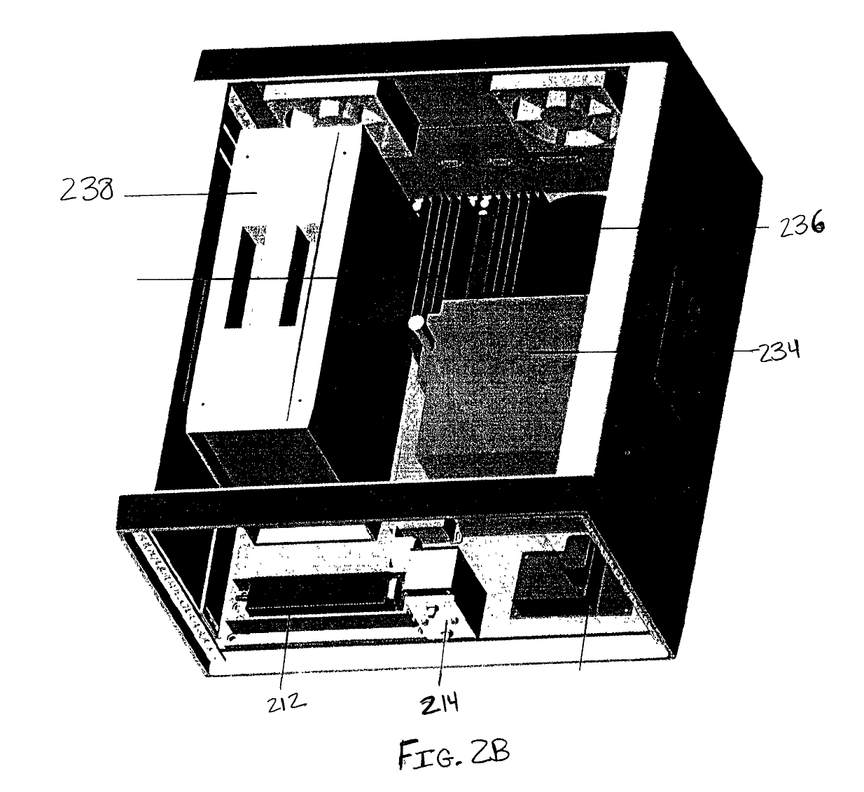

FIG. 2A schematically shows an exemplary embodiment of an apparatus according to the present invention. A detection system 210 includes a laser 212. Laser 212 is preferably a frequency tripled NdYag laser having an emission wavelength of about 355 nm and an average power of approximately 2 milliwatts and is arranged so that its beam illuminates the sample in a cell 214 when a shutter 216 is opened. Cell 214 can be a flow-through cell in which the sample flows through the cell while illuminated. Alternatively, cell 214 can be a simple cuvette. A beam splitter 218 may be provided between laser 212 and shutter 216 to reflect a portion of the beam to a controller 220. Controller 220 comprises a light detector and an electronic circuit to measure the average power output of laser 212, circuitry to open and close shutter 216. Controller 220 is connected to a computer 222, which receives output from controller 220 and sends commands to controller 220. Thus, computer 222 can be any processor that is capable of sending and receiving signals.

A first mirror 224, or alternatively a first mirrored side of cell 214, arranged to reflect the laser beam that has passed through cell 214 back towards laser 212, thereby approximately doubling the effective excitation energy. A second mirror 226, or alternatively a second mirrored side of cell 214, is arranged direct radiation emitted from the sample towards coupling optics 228. In order to filter out radiation at the laser wavelength to ensure that scattered radiation from laser 212 does not interfere with the emission originating from the sample, a barrier filter 230 may be disposed between cell 214 and coupling optics 228. Barrier filter 230 is a high pass filter fabricated so as to remove radiation at the laser wavelength while passing with higher efficiency radiation at higher wavelengths, up to approximately 600 nm. Coupling optics 228 collect radiation emitted by the fluorescing sample and focus the emitted radiation into an optical fiber bundle 232 which transmits the radiation to a spectrometer 234. If cell 214 is more directly coupled to spectrometer 234, fiber bundle 232 can be eliminated. Spectrometer 234, together with a CCD (charge-coupled device) camera 236 and a measurement controller 238, measure the spectrum of the emitted radiation, i.e., the intensity as a function of wavelength, and transmits this data to computer 222. It should be noted that other devices that measure emitted or absorbed radiation can be used in place of the spectrometer/CCD camera arrangement.

Computer 222 analyzes the collected data to determine the disease status of the sample in cell 214. As described in more detail below, computer 222 preferably applies algorithms based on databases comprising spectra of a large number of known positive and negative samples to thereby determine the disease status of the sample in cell 214. In order to facilitate the handling of multiple samples, system 210 can be provided with an automatic sample handler 238 that introduces the sample to be measured into cell 214. Sample handler 238 can also be programmed to rinse cell 214 between introduction of different samples. Preferably, sample handler 238 is under the control of computer 222.

As shown in FIG. 2B, system 210 is preferably provided in a self-contained unit. The unit simply requires connection (either hard-wired or through remote control signals) to computer 222 and, if desired, sample handler 238. Thus, system 210 is portable and provides the user with significant ease and flexibility as to set up and use.

In accordance with the present invention, the absorption and/or fluorescence spectrum of a sample from an individual is used to determine if that person is infected with certain diseases, such as HIV, Hepatitis A, B and C. One convenient sample to use is plasma or other blood fluids. Human plasma contains as many as 100 to 125 proteins, many of which, such as albumin and the lipoproteins, serve as carriers for smaller metabolically important molecules, as well as their metabolites. Applicants of the present invention discovered that the emission spectrum between about 380 nm and about 600 nm can be ascribed to a limited number of these constituents, and these constituents, when present in different relative concentrations, result in a spectrum in which the total fluorescence and emission maximum is shifted to relatively shorter wavelengths for HIV positive plasmas, and to longer wavelengths for HIV negative plasmas.

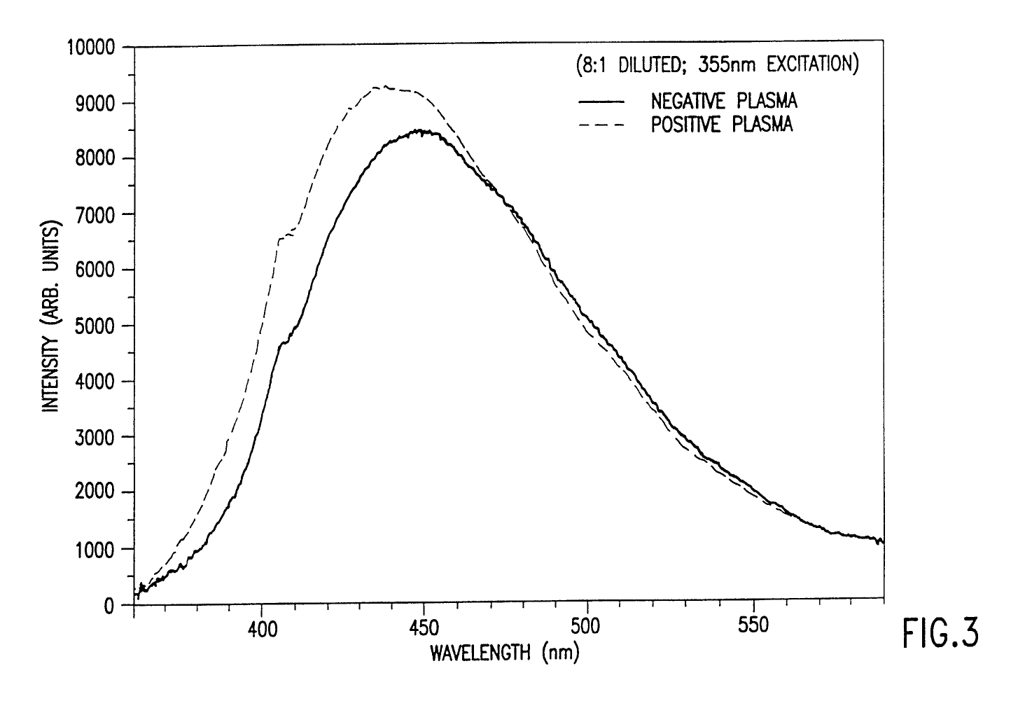

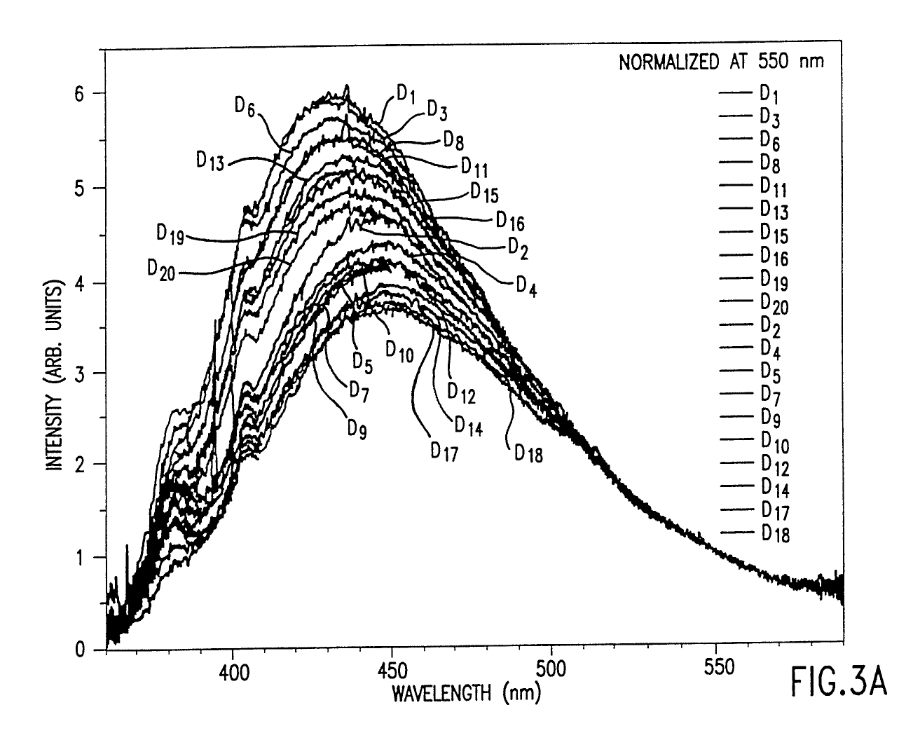

Referring to FIG. 3, which shows the emission spectra of HIV negative (a solid line) and positive (a broken line) whole plasma samples, it is demonstrated that the fluorescence spectra for the HIV negative plasma and HIV positive plasma differ with respect to shape and magnitude. One such difference is that positive samples show increased fluorescence a lower wavelengths. This is further demonstrated by the emission spectra of HIV positive plasmas (D1, D3, D6, D8, D11, D13, D15, D16, D19 and D20) and emission spectra of HIV negative samples (D2, D4, D5, D7, D9, D10, D12, D14, D17 and D18) shown in FIG. 3A. There is a clear separation between the HIV positive spectra group and the HIV negative spectra group.

In accordance with the present invention, methods are provided to alter both the amplitude and wavelength maxima of the plasma spectrum to further discriminate HIV positive and negative samples. It is believed that these methods alter the relative concentration of the contributing fluorophores (the constituents that give rise to the observed fluorescence), which results in the shift in the amplitude and wavelength maxima of the samples. These findings are supported by the following experiments or tests conducted by Applicants.

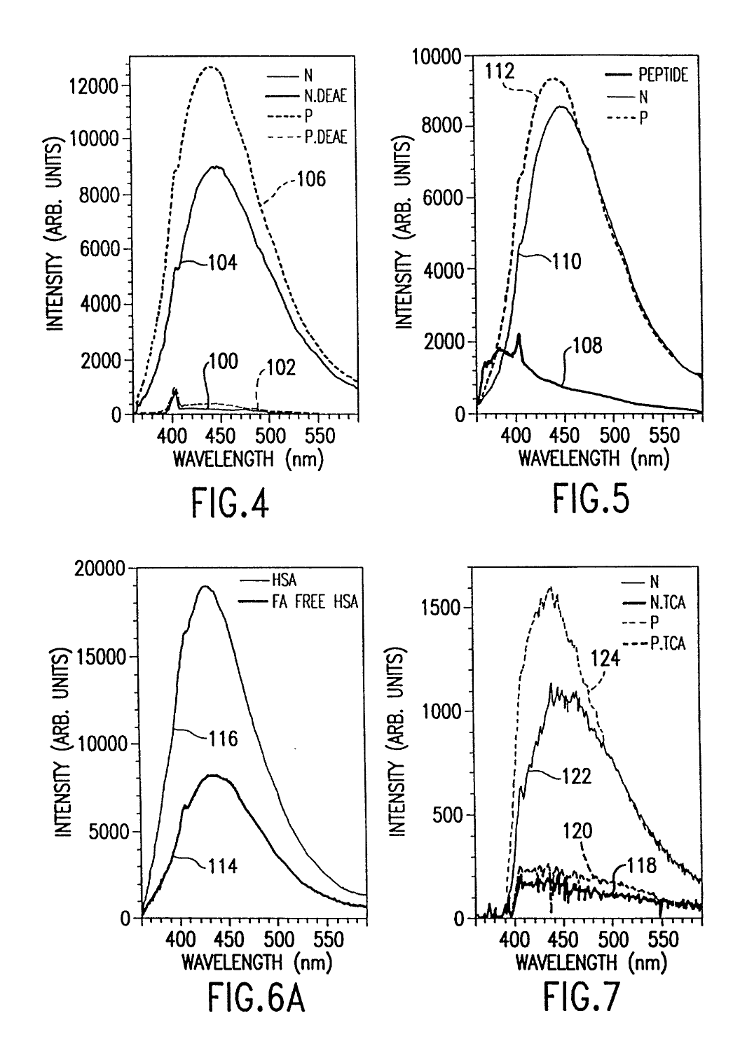

Referring to FIG. 4, the fluorescence emission of eluates obtained by chromatographic absorption of plasma on DEAE-Affi Gel Blue (BioRad Laboratories) essentially yields fractions of purified immunoglobulins. This shows that the immunoglobulins contain less than 2 percent of the fluorescent signal from whole plasma, indicating that the emission profile of the plasma is not due to immunoglobulins. FIG. 4 shows the emission spectrum of eluates made by chromatographic absorption of an HIV negative sample (designated as 100—a thick solid line); the emission spectrum of eluates made by chromatographic absorption of an HIV positive sample (designated as 102—a thick broken line); the emission spectrum of a whole plasma of an HIV negative sample (designated as 104—a thin solid line); and the emission spectrum of a whole plasma of an HIV positive sample (designated as 106—a thin broken line).

Referring to FIG. 5, Applicants found that purified synthetic polypeptides containing 18 of the most commonly occurring amino acids, including phenylalanine, tyrosine, proline and tryptophan, fail to give a significant emission spectra, which indicates that the emission profile of whole plasma in the wavelength range of about 350 nm to about 600 nm is not directly due to a protein or polypeptide. FIG. 5 shows the emission spectrum of the purified synthetic polypeptides (designated as 108—a thick solid line); the emission spectrum of an HIV negative whole plasma sample (designated as 110—a thin solid line); and the emission spectrum of an HIV positive whole plasma sample (designated as 112—a broken line).

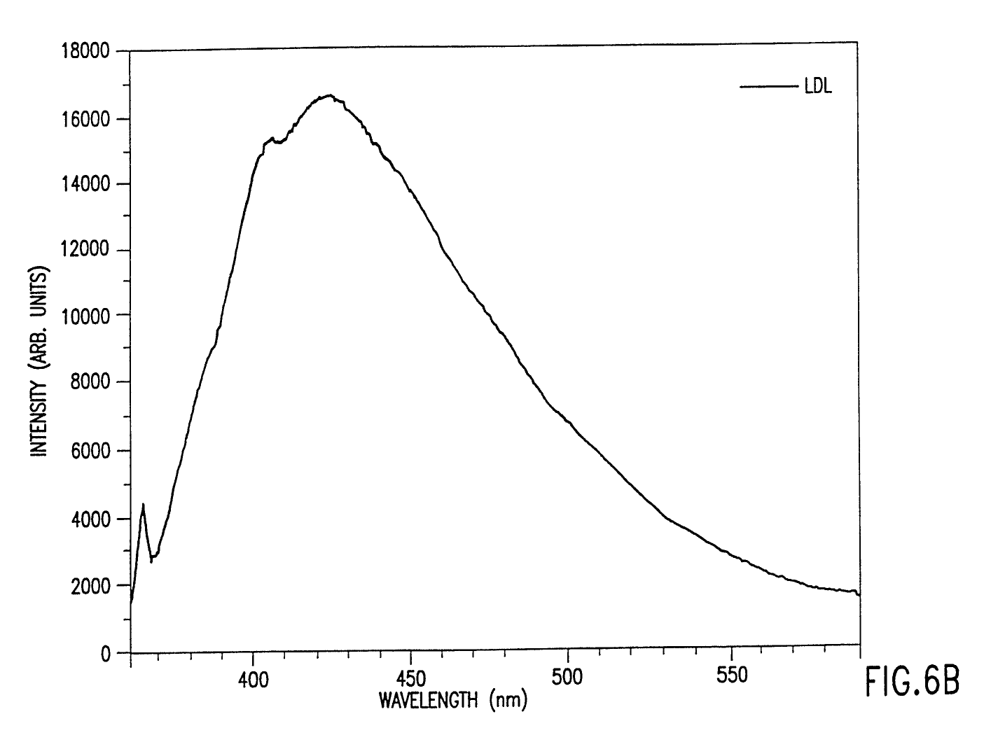

Referring to FIGS. 6A and 6B, analysis of commercially obtained human serum albumin (HSA) (FIG. 6A) and low density lipoprotein (“LDL”)(FIG. 6B) shows that each yields an emission profile very similar to that obtained from whole plasma. In FIG. 6A, the emission spectrum designated as 114 (a thick solid line) is obtained from a fatty acid free HSA sample; and the emission spectrum designated as 116 (a thin solid line) is obtained from an HSA sample. Since purified polypeptides yield virtually no emission signal, Applicants concluded that the HSA and LDL emission signals result from molecules bound to them, but not due to the protein itself. This is in part confirmed by Applicants by taking the fluorescence spectrum of albumin commercially treated with charcoal to remove lipids and other relatively hydrophobic molecules (“essentially fatty acid free albumin”)( see emission spectrum designated as 114 of FIG. 6A): the resulting spectrum showed an emission which has a much reduced intensity relative to that of holo-albumin. From these results, it is concluded that the observed shift in the emission spectra suggests the removal by charcoal of some fluorophores which may contribute to a positive (with HIV viruses) or negative (no detectable HIV viruses) spectra. Similar tests by Applicants with LDL adsorbed by charcoal show a significant decrease in total fluorescence as well as a shift in the spectrum, which suggests that charcoal removes mainly those components bound to LDL that contribute to the emission profile. It is therefore concluded by Applicants that charcoal shifts negative HIV plasma more than HIV positive plasma.

Referring to FIG. 7, plasma in which proteins were precipitated out using trichloroacetic acid (“TCA”) yields a fluorescence spectrum showing a significantly reduced emission intensity relative to that of whole plasma. Applicants thus confirmed that the source of the emission spectra in the wavelength range of interest may be bound to proteins including albumin and LDL. In FIG. 7, the emission spectrum designated as 118 (a thick solid line) is obtained from an HIV negative plasma sample treated with TCA; the emission spectrum designated as 120 (a thick broken line) is obtained from an HIV positive plasma sample treated with TCA; the emission spectrum designated as 122 (a thin solid line) is obtained from the HIV negative plasma; and the emission spectrum designated as 124 (a thin broken line) is obtained from the HIV positive plasma sample.

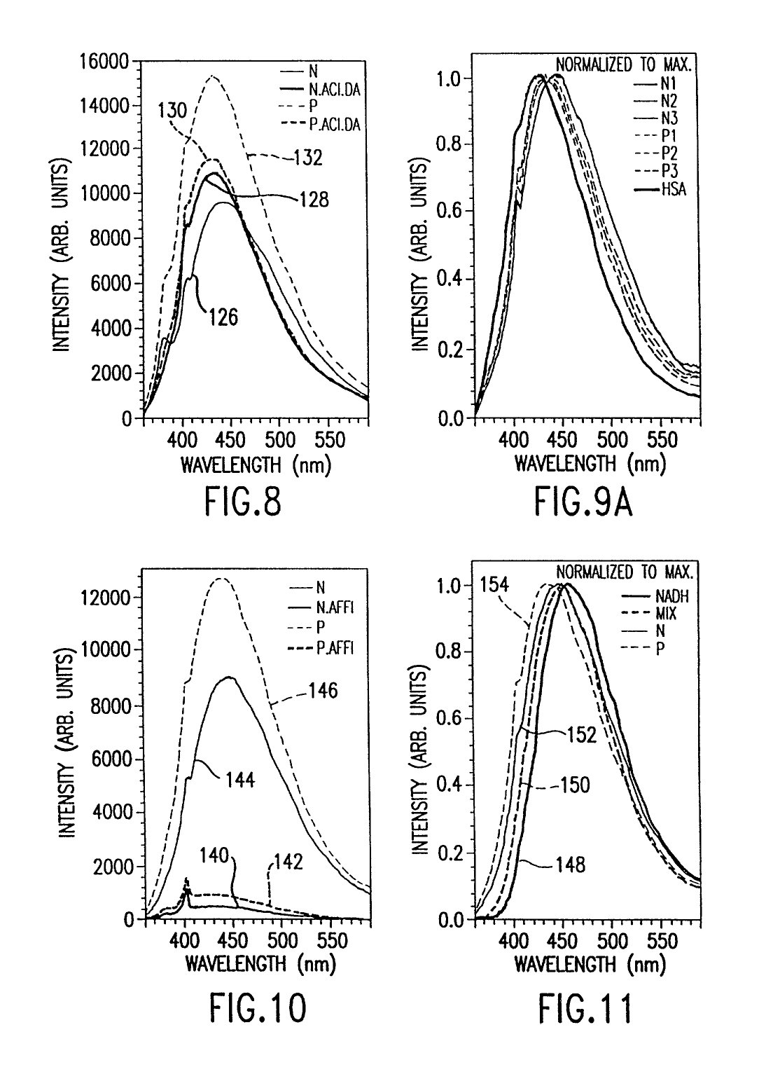

Referring to FIG. 8, the hydrophobicity of these constituents or the hydrophobic rather than the ionic nature of their binding to a protein was demonstrated by the results of experiments which showed little change in spectra upon changing the charge on plasma proteins with acidification to pH 4.0 and subsequent dialysis using membranes with a molecular weight cutoff of 12 kilodaltons. In FIG. 8, the emission spectrum designated as 126 (a thin solid line) is obtained from an HIV negative plasma sample; the emission spectrum designated as 128 (a thick solid line) is obtained from the same HIV negative plasma sample treated by the above described acidification and dialysis process; the emission spectrum designated as 130 (a thick broken line) is obtained from an HIV positive plasma sample treated by the above described acidification and dialysis process; and the emission spectrum designated as 132 (a thin broken line) is obtained from the same HIV positive plasma sample treated by the above described acidification and dialysis process.

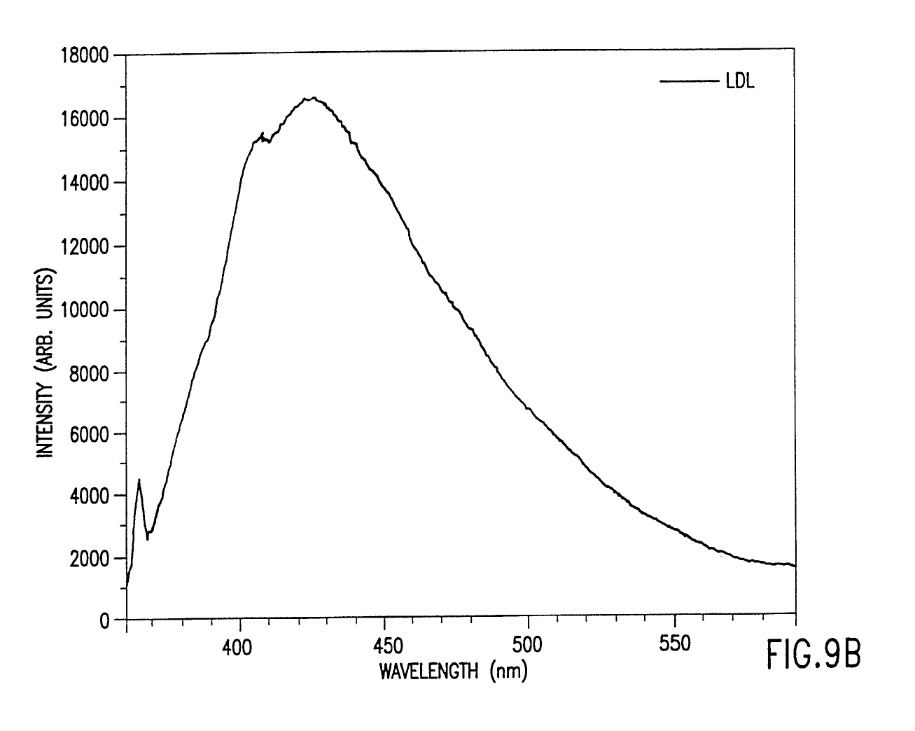

Referring to FIGS. 9A and 9B, the spectra of commercially obtained holo-albumin and LDL closely approximate the spectrum of human plasma. This indicates that a large proportion of the constituents which contribute to the overall emission profile are bound to albumin and LDL.

Referring to FIG. 10, it has been discovered by Applicants that all formulations of CM-Affi Gel Blue (AGB) columns, if used in a high enough ratio relative to plasma, will remove some or all of the fluorescence emission signal. This suggests that this gel has affinity for at least some or all of the fluorophores present in the sample plasma. Cibacrom gels, which contains the same Cibacrom dyes that in CM-AGB, bind other proteins as well, and one common feature of many these proteins is their ability to bind to NADH. It is believed that NADH may acts as a contributing fluorophore in the plasma samples. In FIG. 10, the emission spectrum 140 (a thick solid line) is obtained from an HIV negative sample plasma treated with AGB; the emission spectrum 142 (a thick broken line) is obtained from an HIV positive sample plasma treated with AGB; the emission spectrum 144 (a thin solid line) is obtained from the same HIV negative sample plasma without the AGB treatment; the emission spectrum 146 (a thin broken line) is obtained from the same HIV positive sample plasma without the AGB treatment.

Referring to FIG. 11, analysis of NADH (reduced nicotinamide adenine dinucleotide) yielded a spectrum which overlaps with the spectrum of whole human plasma derived from a negative plasma, also referred to in this application as a “normal” plasma.

Applicants found that the oxidized form, NAD, has no fluorescence in the 380-600 mm wavelength range so that oxidation of a sample resulting in the conversion of NADH to NAD could lead to false positives. Subsequent studies mixing NADH with “essentially fatty acid free” (charcoal treated) albumin gave an emission spectrum which even more closely resembled the negative plasma's emission profile. These results indicate that NADH, possibly in a protein bound form, is an important contributor to the plasma's emission profile. In fact, it has been documented that the presence of HIV viruses results in a reduction in niacin and NADH through the effects of interferon gamma's induction of the catabolism of tryptophan, the obligatory precursor to niacin. In FIG. 11, an emission profile designated as 148 (a thick solid line) is obtained from NADH; an emission profile designated as 150 (a thick broken line) is obtained from a mixture of NADH and HSA; an emission profile designated as 152 (a thin solid line) is obtained from an HIV negative sample plasma; and an emission profile designated as 154 (a thin broken line) is obtained from an HIV positive sample plasma.

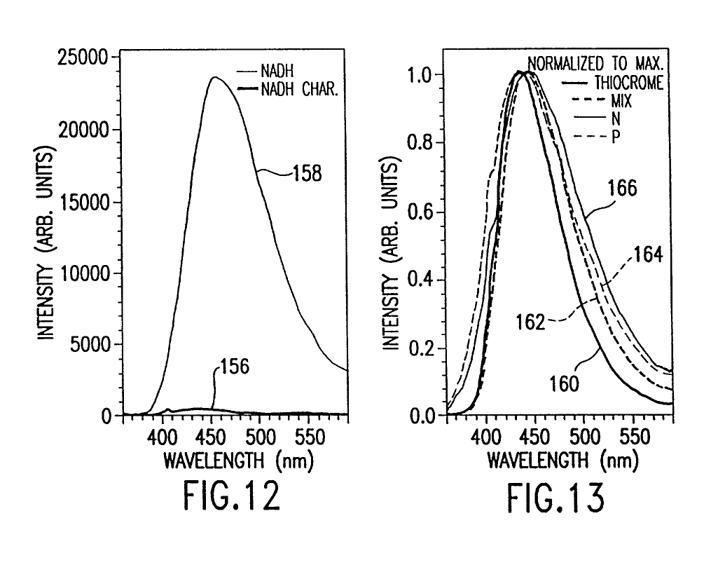

Referring to FIG. 12, additional studies by Applicants have served to reinforce the importance of NADH. Chromatography of plasma through charcoal results in a large reduction in fluorescence while a similar exposure of NADH to charcoal quantitatively removes its signal. In a complex mixture of fluorophores, the relatively selective removal of some fluorophores, such as NADH, would result in a shift in the wavelength of maximum absorption of the aggregate emission spectrum. NADH emits on the long wavelength side of the plasma emission maximum. Thus, any treatment that results in the relatively selective removal of NADH is expected to lead to an overall shift of the emission band towards shorter wavelengths. Applicants found this to be true for all plasma treated with activated charcoal. Moreover, Applicants found that a charcoal-treated HIV negative plasma exhibits a greater shift of the fluorescence maximum towards shorter wavelengths than a charcoal-treated positive plasma sample, which suggests that NADH or related molecules may have a greater contribution to the emission arising from a negative plasma sample than to that arising from a positive. In FIG. 12, an emission profile designated as 156 (a solid thick line) is obtained from a charcoal treated NADH sample; and an emission profile designated as 158 (a thin solid line) is obtained from an NADH sample.

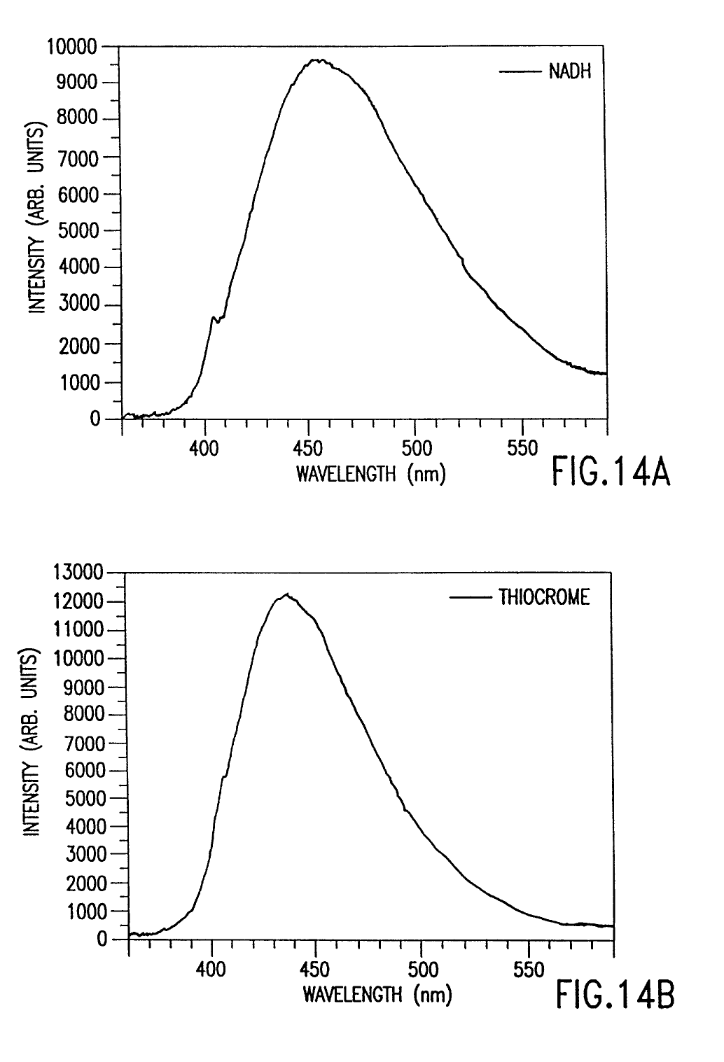

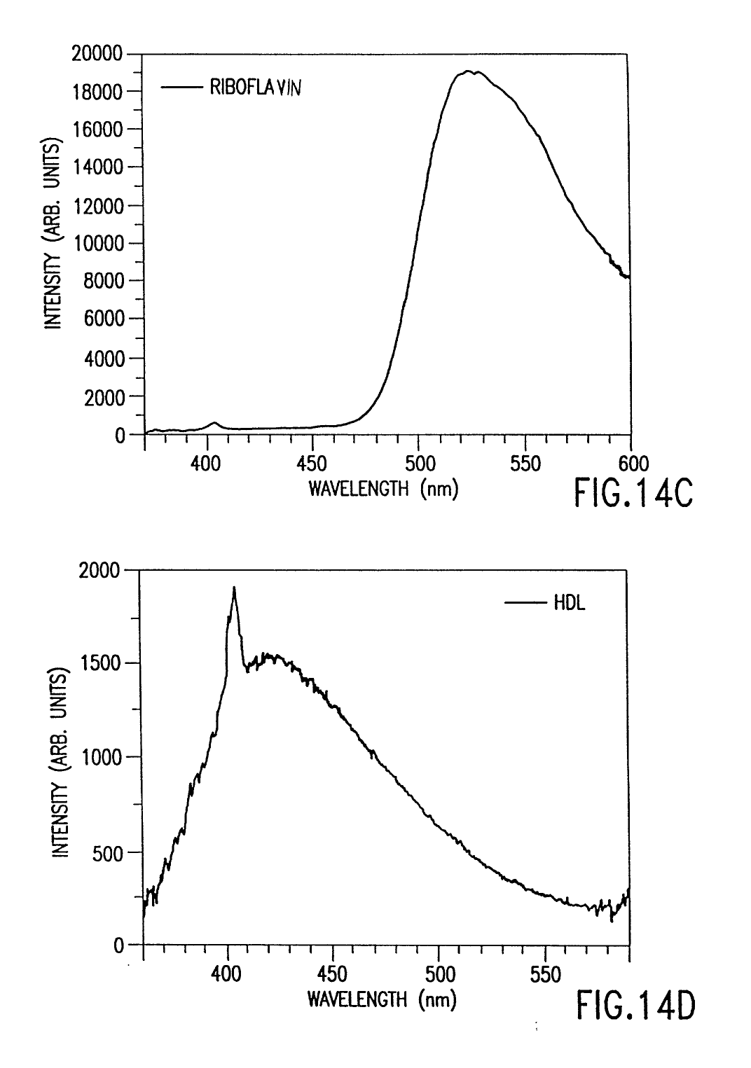

Referring to FIG. 13, analysis of the thiamine metabolite, thiochrome found in plasma, yielded an emission spectrum with a maximum very similar to those of HIV positive samples. FIG. 13 shows the emission profiles obtained, respectively, from thiochrome (designated as 160, a thick solid line), a mixture of thiochrome, NADH and HSA with certain proportions (designated as 162, a thick broken line), an HIV positive plasma sample (designated as 164, a thin broken line), and an HIV negative plasma sample (designated as 166, a thin solid line). It is shown that the mixed sample containing NADH, thiochrome and albumin yielded a spectrum similar to that of a whole plasma sample. These data suggest that these metabolites (NADH, thiochrome, and albumin), in both free and bound forms, make important contributions to the emission spectrum of a human plasma. For completeness, the emission profiles of samples of NADH, thiochrome, riboflavin and HDL are shown in FIGS. 14A-D, respectively.

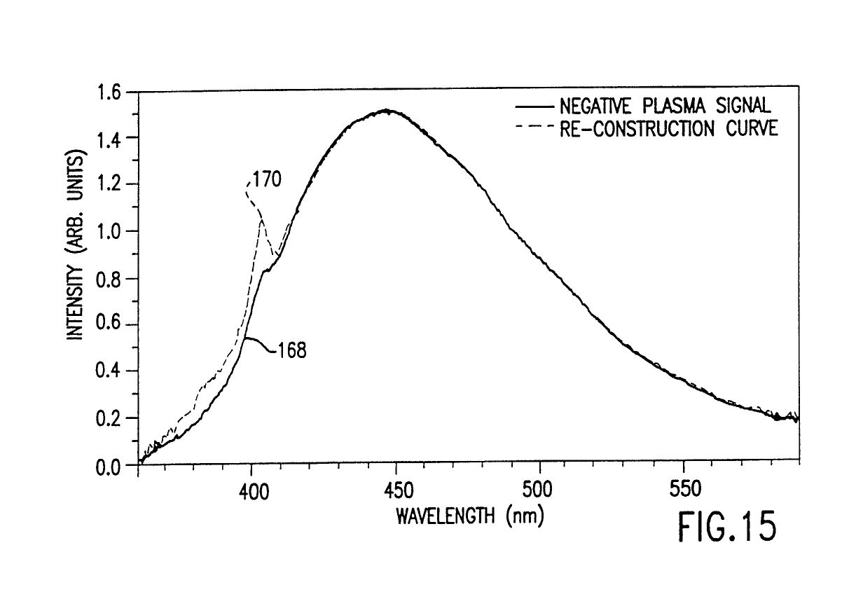

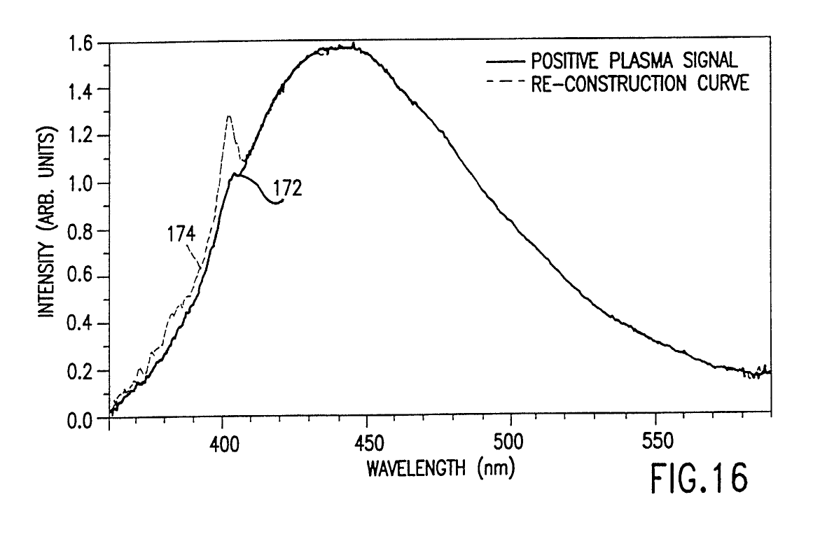

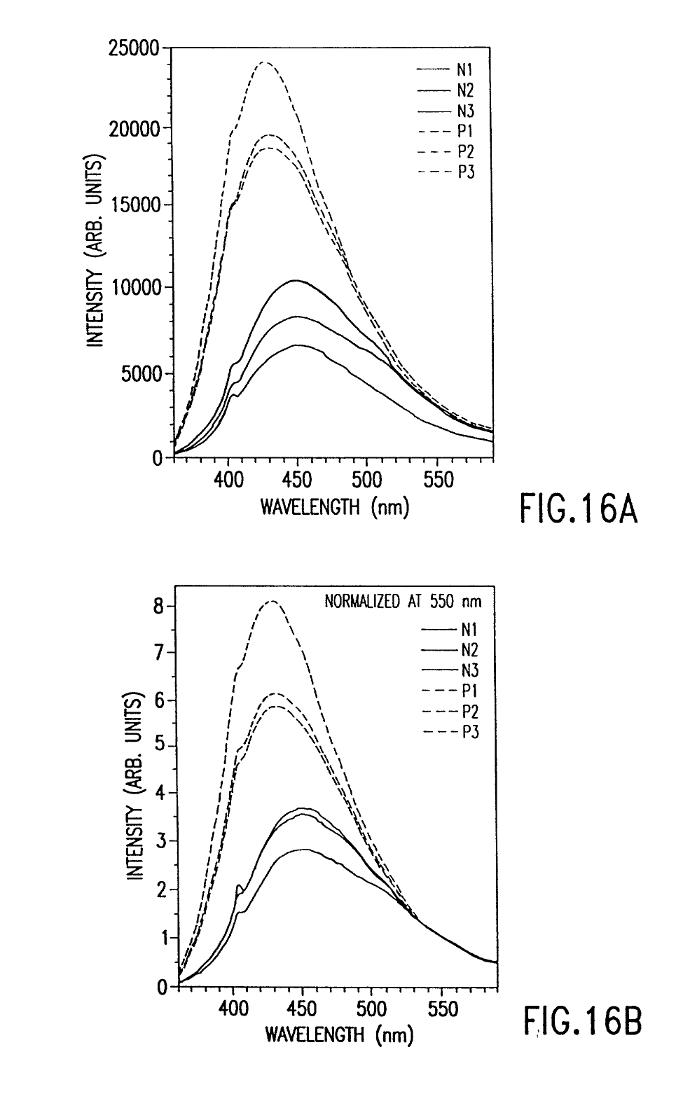

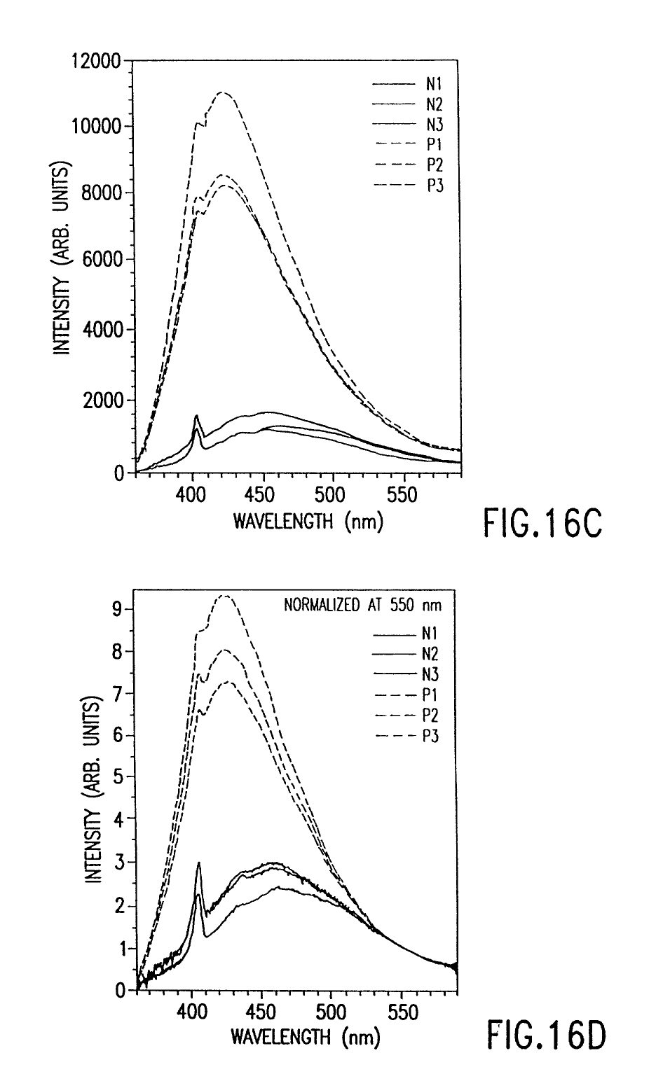

In addition, these data allow one to determine dynamic conditions in which an individual's metabolic status can be assessed based on steady state changes in the relative amounts of these metabolites and similar molecules. As an example, alterations in the relative contributions of the aforementioned metabolites may be made to coincide with either HIV positive or negative plasma, the extent of alteration required thus providing an indication of the relative amounts of metabolites present in the HIV positive or negative plasma. FIG. 15 shows the emission profile of an HIV negative plasma sample (designated as 168—a thin solid line), and that of a sample having a mixture of NADH, thiochrome, and albumin having proportions so that its mission profile (designated as 170—a thin broken line) approximately matches that of the negative HIV plasma sample. FIG. 16 shows the emission profile of an HIV positive plasma sample (designated as 172—a thin solid line), and that of a sample having a mixture of NADH, thiochrome, and albumin having proportions so that its mission profile (designated as 174—a thin broken line) approximately matches that of the positive HIV plasma sample.

Referring to FIGS. 16A-D, Applicants found that C-M (carboxy-methyl) Affi Gel Blue has a differential effect on HIV positive and negative plasmas. These results are obtained when specific ratios of plasma to C-M Affi Gel Blue are used. More specifically, in preparing CM-ABG treated samples, 0.25 milliliter (ml) of plasma is diluted to 2.0 ml with 20 mM of phosphate buffer and 1.5 ml of CM-AGB, which yields consistent and noticeable shifts in the spectra. For example, FIG. 16A shows the emission profiles of three HIV positive plasma samples (broken lines designated as P1, P2 and P3 sequentially from the top most broken line) and three HIV negative plasma samples (solid lines N1, N2 and N3 from the top most solid line), all of which are whole plasma samples as control samples and are untreated with CM-ABG. FIG. 16B shows the same emission profiles of these samples normalized at 550 nm. In comparison, referring to FIG. 16C, the emission profiles of CM-AGB treated HIV positive plasma samples (broken lines P1, P2 and P3) and that of CM-AGB treated HIV negative plasma samples (solid lines N1, N2 and N3) show much more differentiation between the HIV positive sample emission profiles and HIV negative sample emission profiles. Similarly, referring to FIG. 16D which show emission profiles of HIV positive samples treated with CM-AGB (broken lines P1, P2 and P3) and emission profiles of HIV negative samples (solid lines N1, N2 and N3), all normalized at 550 nm, the emission profiles for the CM-AGB treated samples normalized at 550 nm exhibit more differentiation between the emission profiles of the HIV positive samples and these of HIV negative samples, than that of the HIV positive and negative samples untreated with CMAGB.

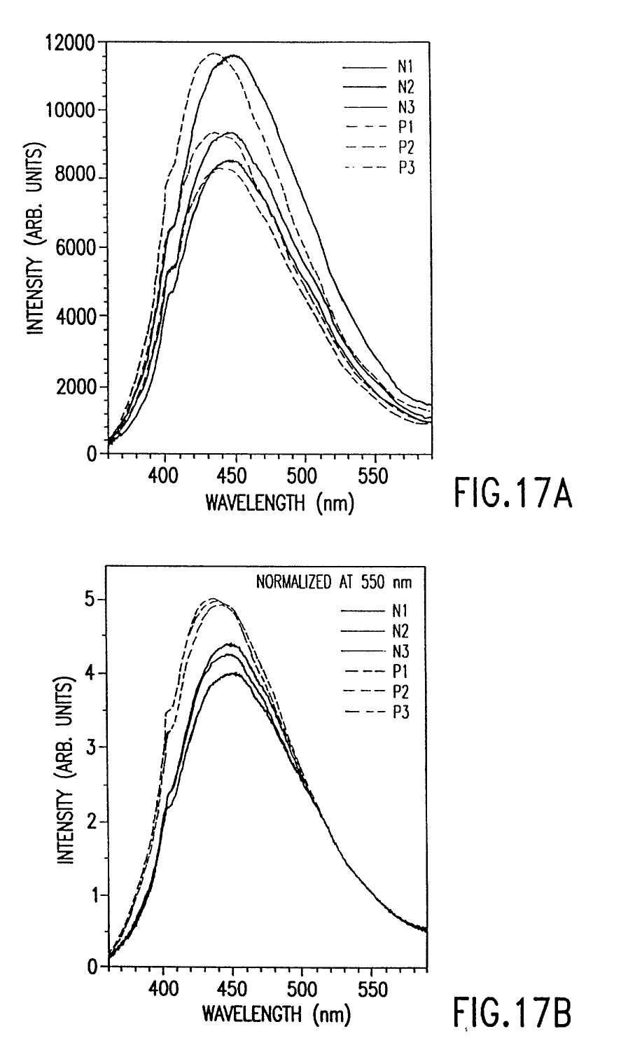

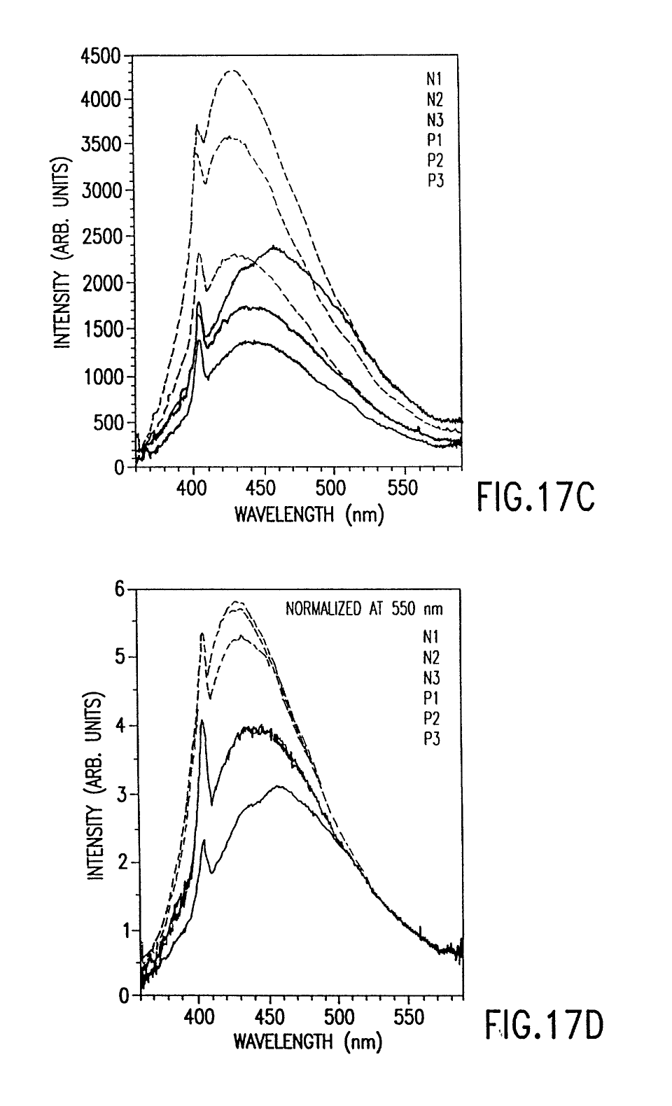

FIGS. 17A-D show emission profiles of another set of six samples, three of which are HIV positive samples and the other three samples are HIV negative samples. FIG. 17A shows the emission profiles of the six samples untreated with CM-AGB (with the three broken lines P1, P2 and P3 for the HIV positive samples, and the three solid lines N1, N2 and N3 for the HIV negative samples). The emission profiles for the same untreated samples normalized at 550 nm are shown in FIG. 17B. The mission profiles for the same samples treated with CM-AGB are shown in FIG. 17C (with the three broken lines P1, P2 and P3 for the HIV positive samples, and three solid lines N1, N2 and N3 for the HIV negative samples). The emission profiles for the same CM-AGB treated samples normalized at 550 nm are shown in FIG. 17D, with the broken lines for the HIV positive samples and solid lines for the HIV negative samples. Again, the emission profiles obtained form the CM-AGB treated samples exhibit a much greater differentiation between the HIV positive and negative samples than that obtained from samples untreated with CMAGB.

In summary, the differential effects of the CM-AGB on HIV positive and negative plasma suggest different plasma constituents have differential affinities for the CM-AGB gel and that these differences, as will be detailed below, are used as markers for HIV positive samples.

In accordance with one embodiment of the present invention, a method is provided to treat the samples before obtaining fluorescence emission spectra from the samples so that the emission spectra, and more particularly, certain characteristics of the emission spectra, from the samples infected with a disease (e.g., HIV viruses) are more differentiable from those samples not infected with such disease. In a preferred embodiment for detecting HIV viruses, the samples are prepared using the following procedures and assay conditions.

The sample is divided into three aliquots. The first aliquot is left untreated and serves as a control sample, designated CT. The second aliquot is chromatographed, preferably using 1.5 ml of carboxy-methyl Cibacron Blue (C-M Affi Gel Blue, Bio Rad Laboratories) and is designated AGB. The third aliquot is adsorbed with 0.2 ml of activated charcoal (Norit A) and designated CH.

Each sample is then analyzed spectrofluorometrically using the system according to the present invention. In accordance with one embodiment of the present invention, spectrofluorimetry yields an emission spectra, the parameters of which is subsequently used to analyze each aliquot of the samples. To ensure the integrity and reproducibility of the emission spectra, Applicants refer all samples to identical levels of radiation energy.

In accordance with the present invention, the analysis of a given sample's emission profile is performed using the parameters defined as follows and the algorithm described in detail below. The definitions of the parameters used in the algorithm are as follows:

λₚ: The wavelength in nanometers at which the peak of fluorescence intensity is obtained. Mathematically, this is when dY/dX=0, where Y is the intensity of fluorescence and X is wavelength of the fluorescence. For example, λ_{p-CT} is the peak wavelength of a control sample.

Am: The amplitude of the peak fluorescence. For example, AMCT corresponds to the amplitude of a control sample.

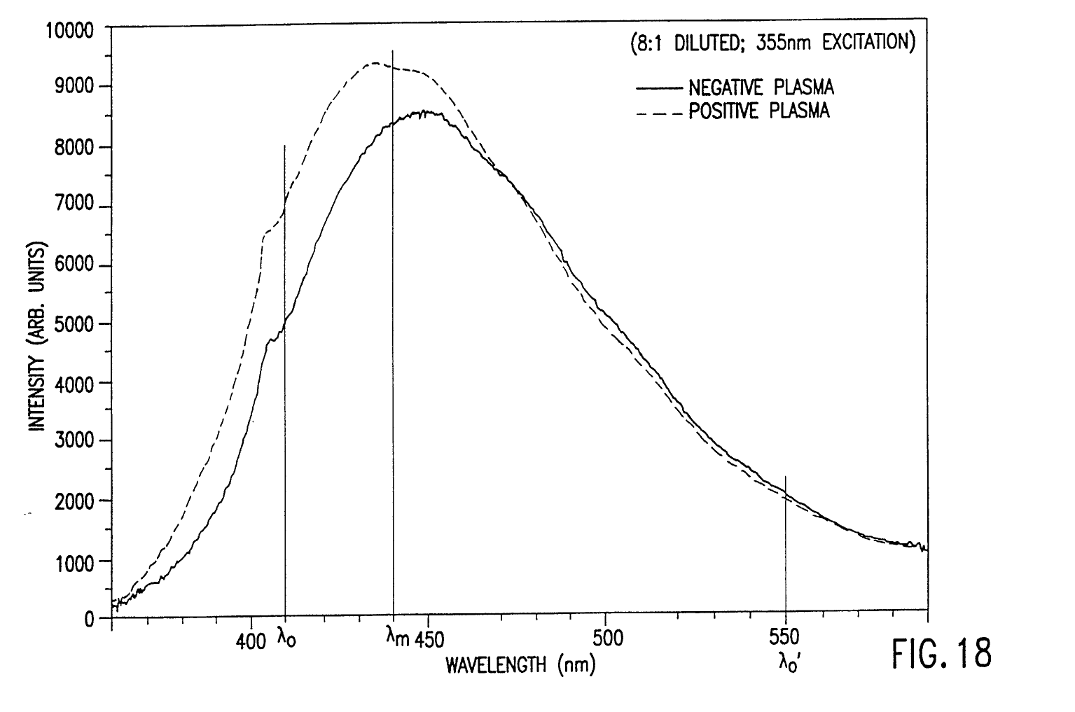

Ar: The area ratio. The area ratio is defined as, as depicted in FIG. 18, the area under the emission spectrum extending from a first selected wavelength (λ₀) to a second selected wavelength (λₘ), divided by the area under the curve extending the second selected wavelength to a third selected wavelength point(λ₀′). Mathematically this will be calculated as [∫_{λ0}^{λm}/∫_{λm}^{λ0′}]. In the preferred embodiment as depicted in FIG. 18, 410 nm, 440 nm and 550 nm are selected as the first, second and third selected wavelengths, respectively. They are also used in all of the area ratio data shown herein. It should be understood that different wavelengths may be used, which may be adapted to obtain the same results as described herein. It should also be understood that analogous parameters for an absorption spectrum can be used if the sample's absorption spectrum is obtained.

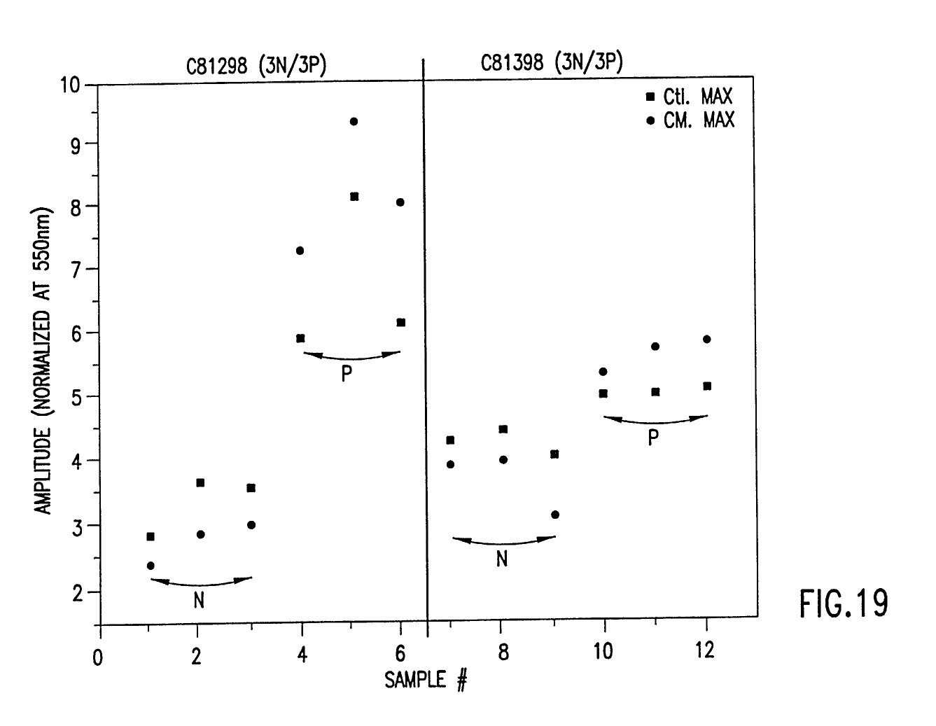

In accordance with the present invention, one or more of these parameters are used to discriminate samples infected with a disease, such as HIV positive samples, from noninfected samples, such as HIV negative samples. For example, referring to FIG. 19, the amplitude, Am, is used to differentiate the HIV positive samples from the HIV negative samples. In FIG. 19, the x-axis corresponds to the sample number, and the y-axis corresponds to the amplitude, Am, of the samples normalized at 550 nm. For each sample, two measurements of the amplitude are performed and shown in FIG. 19, one for the untreated sample (i.e., control sample) which is shown as a back square, the other for the sample treated with CM-AGB which is shown as a black circle. The measurements results designated “N” are obtained from known HIV negative samples, which are determined by commercially available, FDA-approved tests. The measurement results designated “P” are obtained from known HIV positive samples, which are determined by commercially available, FDA-approved tests. As shown in FIG. 19, sample Nos. 1, 2, 3, 7, 8 and 9 are known HIV negative samples, whereas sample Nos. 4, 5, 6, 10, 11 and 12 are known HIV positive samples.

On the basis of the amplitude measurement results shown in FIG. 19, it is seen that the HIV negative samples and HIV positive samples can be discriminated on the basis of their amplitudes, particularly the amplitudes (indicated as black circles in the FIG.) of the CM-AGB treated samples. For example, if a normalized amplitude value of 5 is used to discriminate the samples treated with CM-AGB (that is, the samples having an amplitude above 5 are deemed to be HIV positive whereas the samples having a normalized amplitude less than 5 are deemed to be HIV negative), it is seen that the six HIV positive samples among the 12 samples tested will be correctly discriminated from the six HIV negative samples. It is noted that the CM-AGB treatment of the samples improves the discrimination of the infected samples from the uninfected samples.

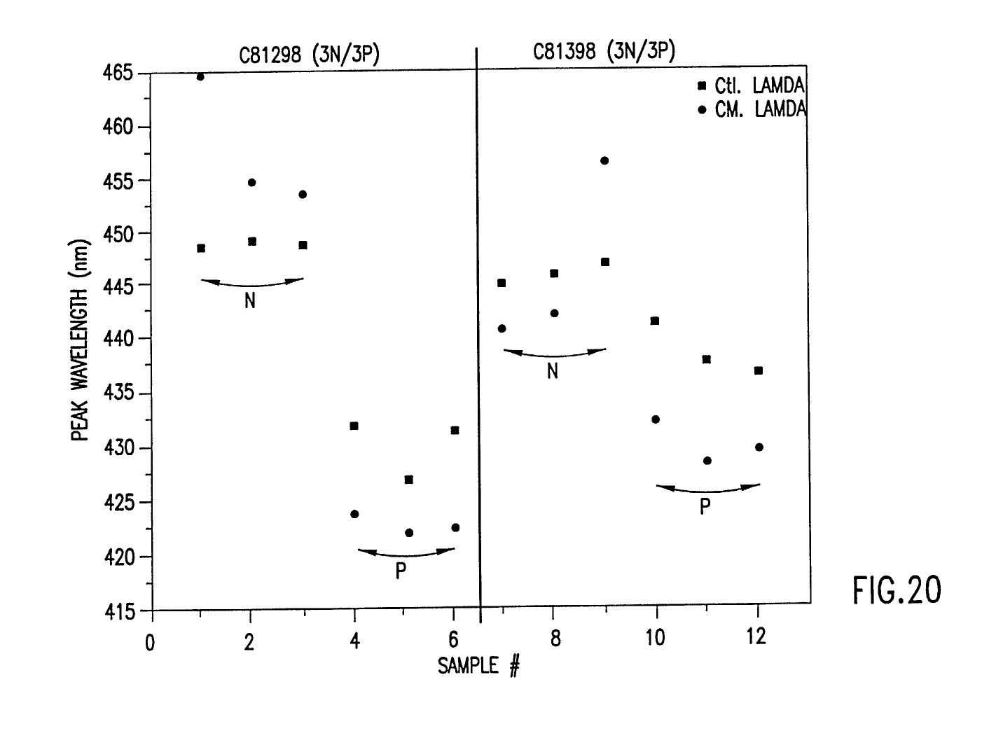

In accordance with the present invention, peak wavelength, λₚ, can be used to discriminate infected samples, such as HIV positive samples, from uninfected samples, such as HIV negative samples. Referring to FIG. 20, which are the peak wavelength measurement results of the same set of samples that are used to obtain the amplitude measurements shown in FIG. 19, and which has the same designations, if a peak wavelength of 435 nm is used to discriminate the CM-AGB treated HIV positive samples from the CM-AGB treated HIV negative samples, it is seen that the six HIV positive samples among the 12 samples tested will be correctly discriminated from the six HIV negative samples. It is also noted that the CM-AGB treatment of the samples improves the discrimination of the infected samples from the uninfected samples for peak wavelength measurement.

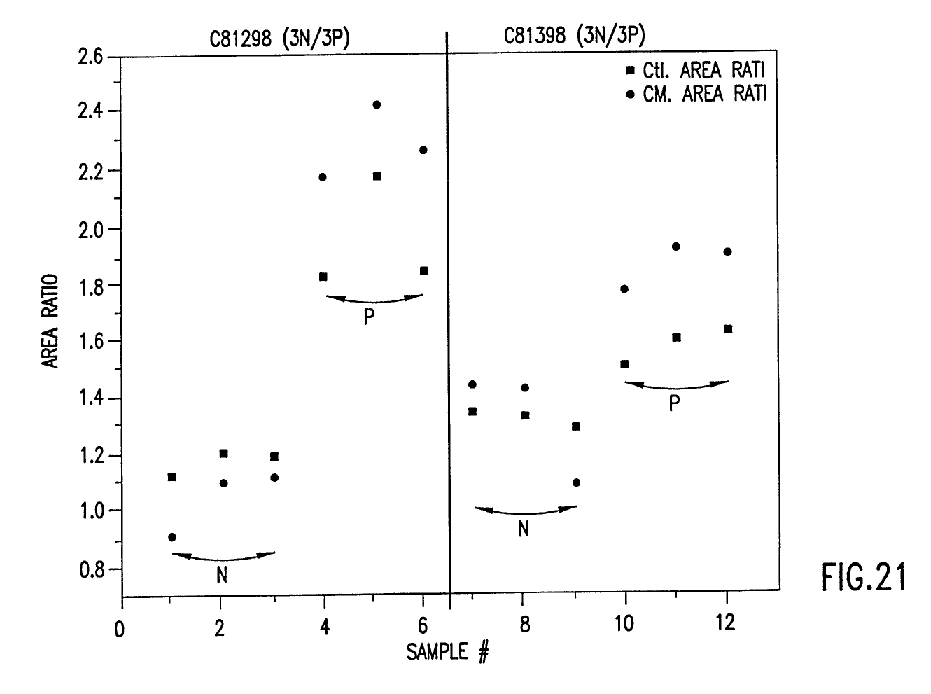

In accordance with the present invention, area ratio, Ar, can be used to discriminate infected samples, such as HIV positive samples, from uninfected samples, such as HIV negative samples. Referring to FIG. 21, which are the area ratio measurement results of the same set of samples that are used to obtain the amplitude measurements shown in FIG. 19, an area ratio of 1.6 is used to discriminate the CM-AGB treated HIV positive samples from the CM-AGB treated HIV negative samples, the six HIV positive samples among the 12 samples tested will be correctly discriminated from the six HIV negative samples. It is clear from the test results that the CM-AGB treatment of the samples improves the discrimination of the infected samples from the uninfected samples.

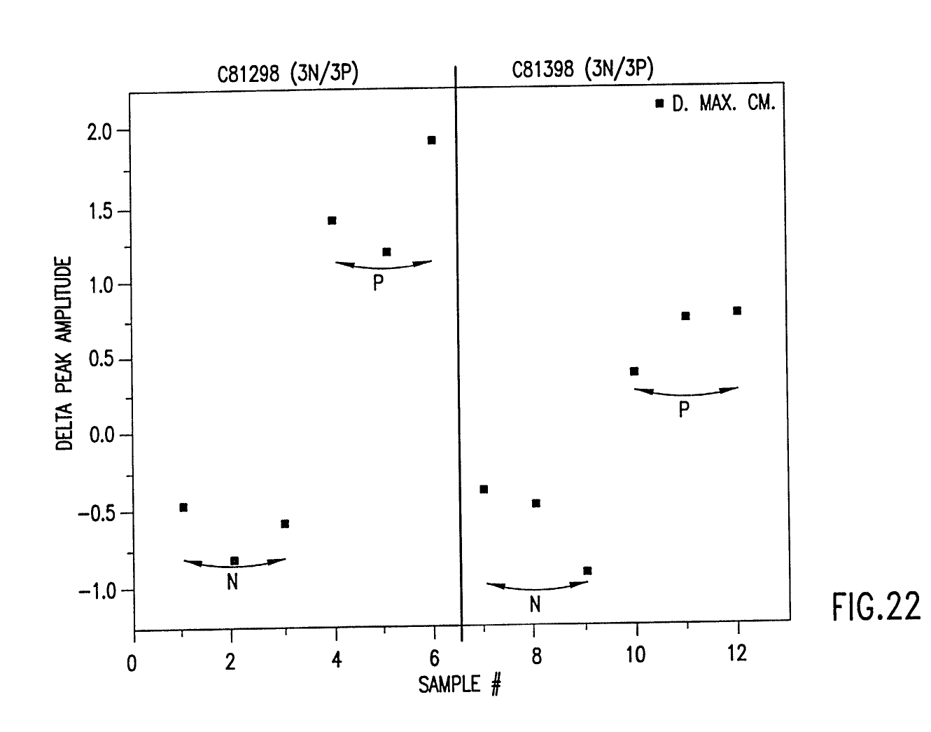

In accordance with the present invention, changes of the amplitudes of samples, ΔAm, before and after CM-AGB treatment, can be used to discriminate infected samples, such as HIV positive samples, from uninfected samples, such as HIV negative samples. Referring to FIG. 22, which shows the amplitude changes for the same set of samples that shown in FIG. 19, if no change of amplitude (i.e., ΔAm=0.0) is used to discriminate the HIV positive samples from the HIV negative samples, it is seen that the six HIV positive samples among the 12 samples tested will be correctly discriminated from the six HIV negative samples.

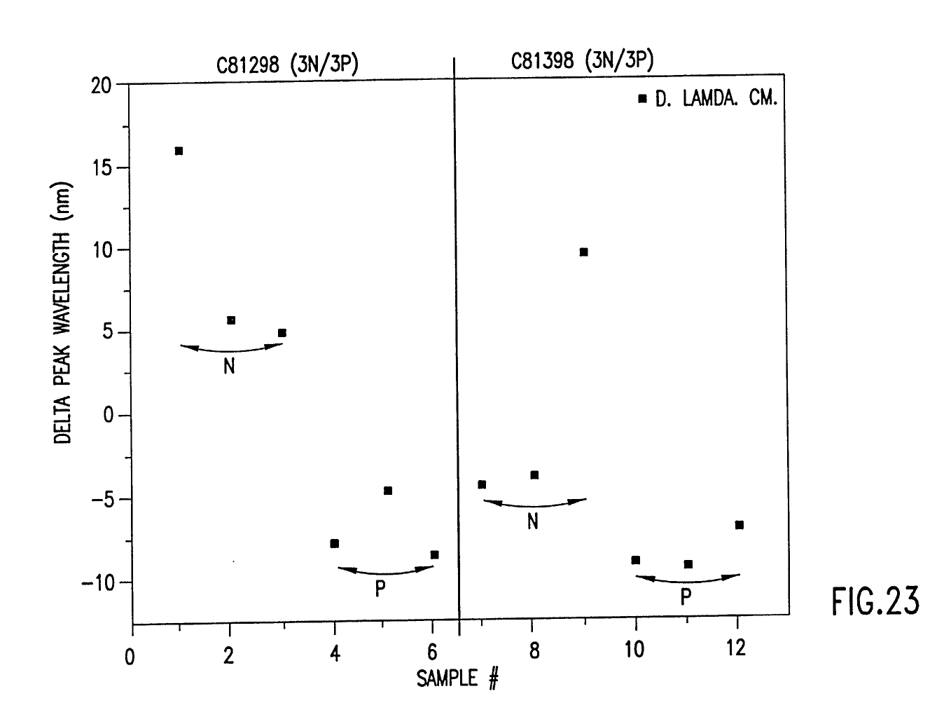

In accordance with the present invention, changes of the peak wavelengths of samples, Δλ_{p,} before and after CM-AGB treatment, can be used to discriminate infected samples, such as HIV positive samples, from uninfected samples, such as HIV negative samples. Referring to FIG. 23, which shows the amplitude changes for the same set of samples in FIG. 19, if no change of peak wavelength (i.e., Δλₚ=0.0 nm) is used to discriminate the HIV positive samples from the HIV negative samples, it is seen that HIV positive sample Nos. 4, 5, 6, 10, 11 and 12 will be discriminated from HIV negative sample Nos. 1, 2, 3 and 9. However, HIV negative sample Nos. 7 and 8 will not be discriminated from the HIV positive samples.

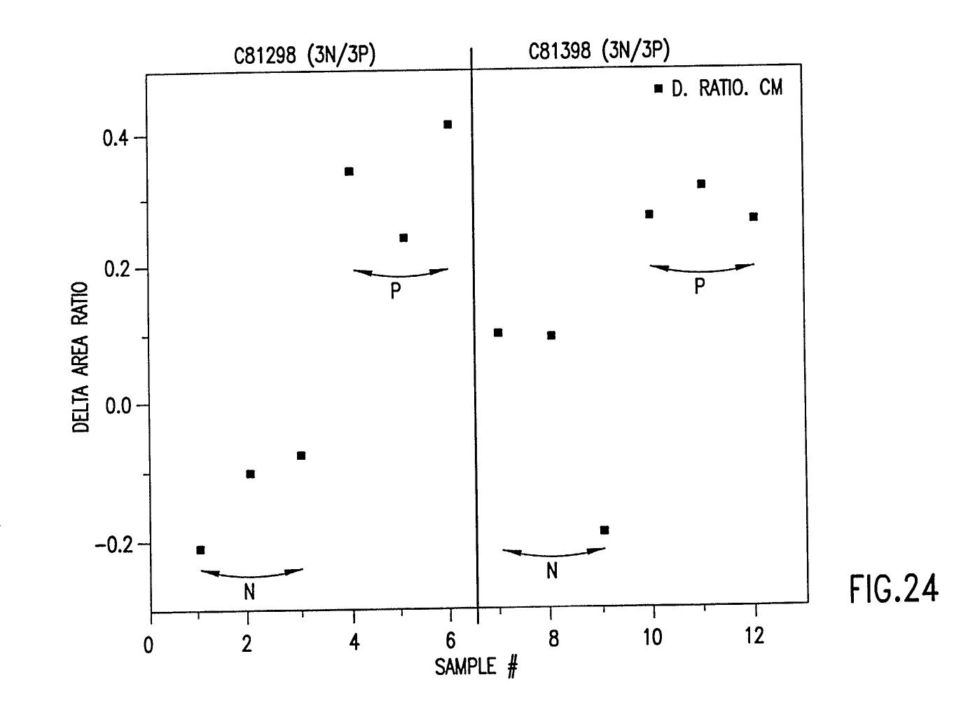

In accordance with the present invention, changes of area ratios of the samples, ΔAr, before and after CM-AGB treatment, can be used to discriminate infected samples, such as HIV positive samples, from uninfected samples, such as HIV negative samples. Referring to FIG. 24, which shows the area ratio changes for the same set of samples in FIG. 19, if no change of area ratio (i.e., ΔAr=0.0) is used to discriminate the HIV positive samples from the HIV negative samples, HIV positive sample Nos. 4, 5, 6, 10, 11 and 12 will be discriminated from HIV negative sample Nos. 1, 2, 3 and 9, but HIV negative sample Nos. 7 and 8 will not be discriminated from the HIV positive samples. However, if a change of area ratio of 0.2 (i.e., ΔAr=0.2) used, all of the HIV positive samples will be discriminated from the HIV negative samples.

In accordance with the present invention, although one of the above-mentioned parameters by itself may not be able to discriminate infected samples from uninfected samples with one hundred percent accuracy, if more than one of the parameters are used, high accuracy may nevertheless be achieved. In addition, proper algorithms using one or more of the above described parameters may be used to discriminate infected samples from non-infected samples.

In accordance with the present invention, various discriminators are provided for discriminating infected samples from non-infected samples.

In a preferred embodiment, a discriminator D1 is constructed from 1/λₚ, Am and Ar, all three of which will be greater for positive samples than negative samples or the mean value from a normal data base. Thus, any additive or multiplicative combination of these parameters from positive samples divided by an identical combination of these parameters from the normal data base will be greater than 1. The value will be closer to 1 or less than 1 for negative samples. Such an analytic function for D1 is expressed as D1=f(1/λₚ, Am, Ar).

Another discriminator D2 is provided, which takes advantage of the differential shifts in these parameters (1/λₚ, Am, Ar) after the sample undergoes chromatography through CM-Affi Gel Blue. After chromatography, Δλₚ is greater for HIV positive samples than for HIV negative (normal) samples. Because ΔAm is greater for HIV negative samples than for HIV positive samples, the reciprocal of the difference in Am, 1/ΔAm, is greater for positive samples. The parameter ΔAr is greater for positive samples. Thus, any additive or multiplicative combination of these parameters from positive samples divided by an identical combination of these parameters from the normal data base will again be greater than 1. This ratio for negative samples will be closer 1 or less than 1. Such an analytic function for D2 is expressed as D2=f(Δλₚ, Δ1/Am, ΔAr).

Another discriminator D3 is provided, which will reflect the differential effects of adsorption by charcoal. The parameter 1/Δλₚ will be greater for HIV negative samples than for HIV positive samples, so will be the parameter 1/ΔAr. Thus, any additive or multiplicative combination of these parameters divided by the same combination from the normal data base will be greater than 1 for positive samples and closer to 1 or less than 1 for HIV negative samples. Such an analytic function for D3 is described by D3=f(Δλₚ, 1/ΔAr).

In accordance with the present invention, an aggregate discriminator D*=f(iD1, jD2, . . . , kD3) can be provided, which will yield values for HIV positive samples greater than for HIV negative samples. The coefficients i through k are weighing factors to be determined empirically. HIV Negative samples will yield a range of D*s, the variance of which within the data base for the normal samples (i.e., HIV negative samples) must be determined. This will be determined from large scale testing by the LBS system of the present invention.

In accordance with the present invention, analyses other the above-identified parameters and discriminators can be used to differentiate spectra of samples from diseased and non-diseased individuals. Specifically, decomposition and/or partial fitting techniques can probe subtle differences in the spectra. One example is wavelet analysis, in which a multilevel decomposition process is applied to the spectral signal to generate a set of curve approximations (low frequencies) and details (middle and high frequencies). Experimental studies with HIV positive plasma samples have shown that common features are found in middle frequencies at some spectra regions. The parameters, amplitude and phase, generating the decomposition can provide variables used for discrimination. The present invention also contemplates the use of other algorithms, such as neural networks, for discrimination of samples from diseased and non-diseased individuals.

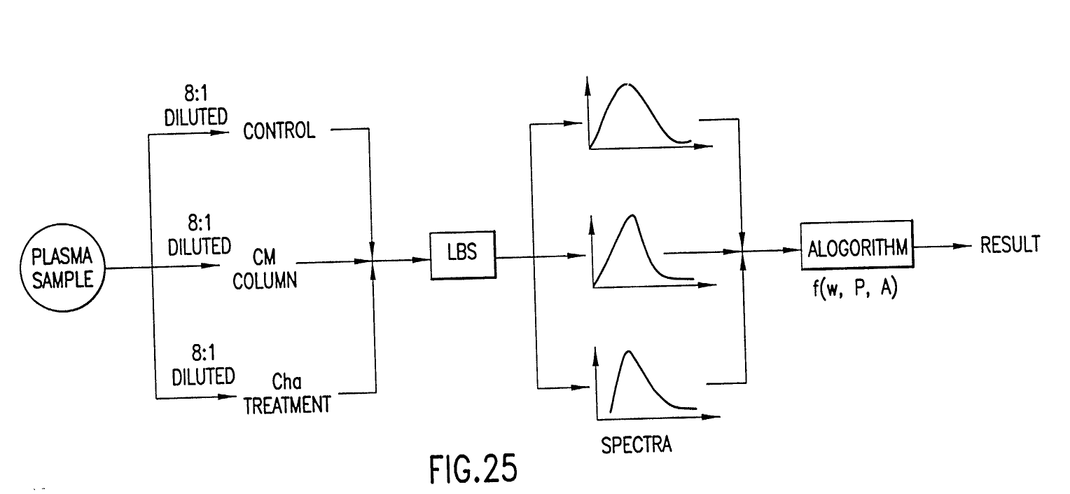

FIG. 25 illustrates a flow diagram showing an exemplary embodiment of the disease detection method of the present invention. A plasma blood sample to be tested is first divided into three portions: the first portion is diluted 1:8 with potassium phosphate buffer and will be used as a control sample (referred as “control”); the second portion is also diluted 1:8 with potassium phosphate buffer and then chromatographed through CM-AGB (referred as “CM Column”); and the third portion is treated with activated charcoal (referred as “Ch Treatment”). All three portions are then provided to the LBS of the present invention to obtain their fluorospectram in the desired wavelength band with excitation at a desired wavelength. Their fluorospectrum and characteristics and/or parameters are then used in an algorithm for calculation, which provides a final indicator as to whether or not the sample is infected with certain infectious diseases.

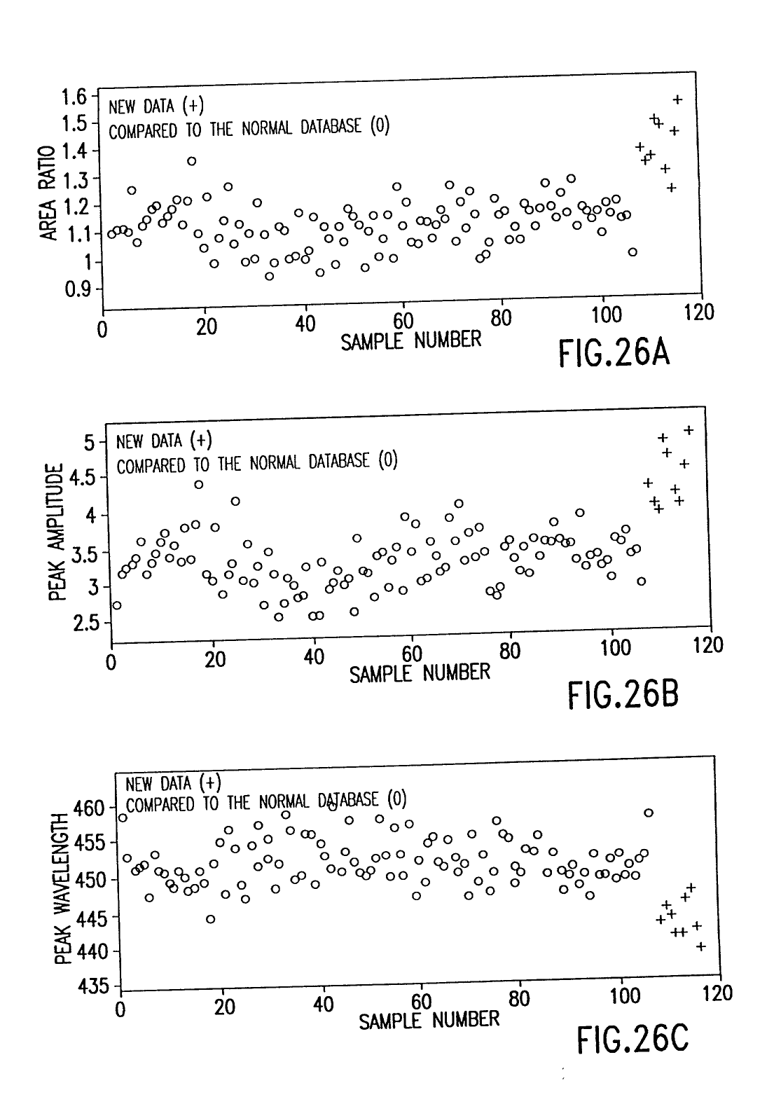

The method and apparatus for infectious disease detection of the present invention may be used to detect HIV infection at a very early stage where it is still not detectable by the widely used enzyme-linked immunosorbent assay method (“the ELISA method”) or conventional clinical diagnosis. FIGS. 26A-C show the measurement results of the area ratio, peak amplitude, and peak wavelength, of the normal database (represented as “o”), respectively, and those of blood samples, untreated with CM-AGB or charcoal, (i.e., control samples) from a single individual at difference times at an early stage of HIV infection (represented as “+”). There are nine samples taken from the same individual and tested and their test results are arranged such that the left-most result is from the blood plasma sample first taken in time, and the right-most result from the blood plasma sample last taken in time, and the results in-between are arranged sequentially from the earlier-taken samples to the later-taken samples. As shown in FIGS. 26A-C, the test results of the blood samples from the HIV infected individual are fairly discriminated from those of the normal database.

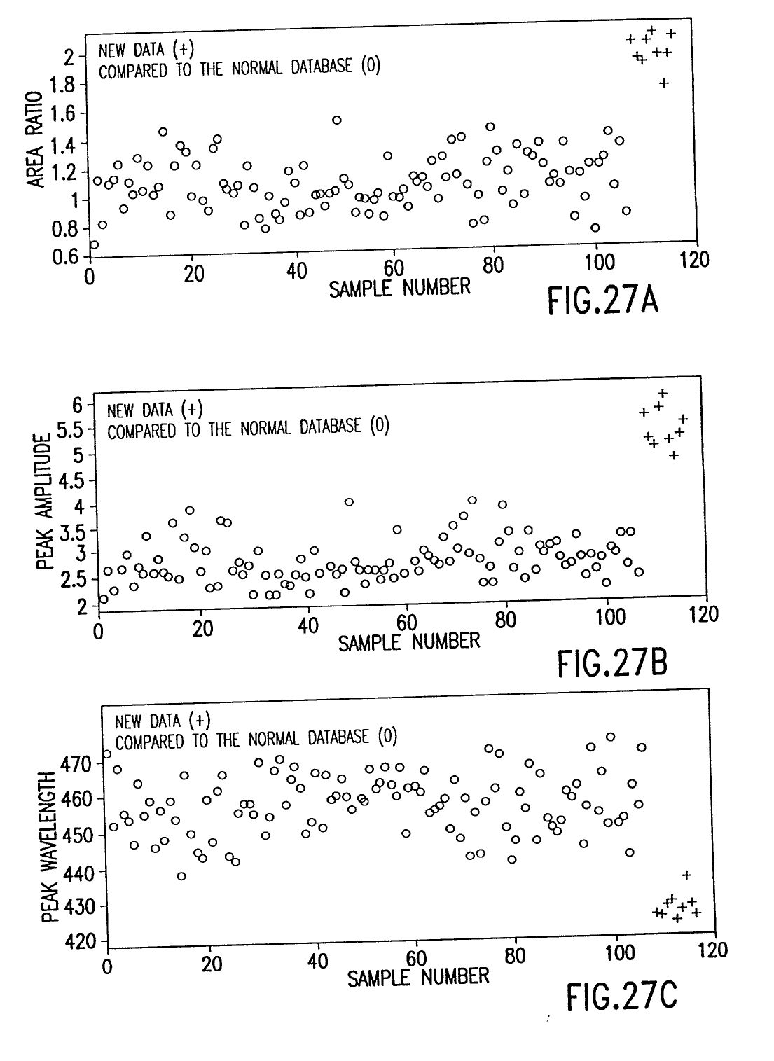

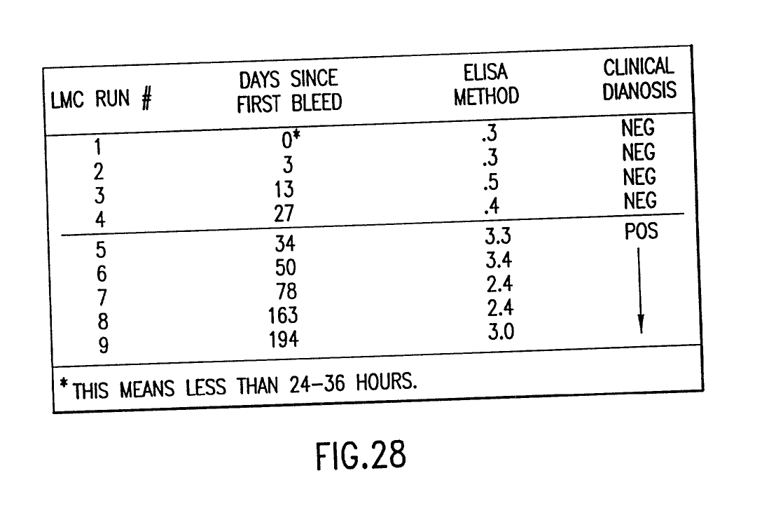

FIGS. 27A-C are the measurement of the same parameters for the same samples after the samples are treated with CM-AGB. As shown, the discrimination between the results of the infected samples and those of the normal database is even more evident than the measurement on the untreated samples; the infected samples are clearly discriminated from the normal database. FIG. 28 is a table listing the sample numbers, the relative time when the samples are taken, and their ELISA method test results, and conventional clinical diagnosis test results. For the first four samples, both the ELISA method and conventional clinical diagnosis fail to detect the HIV viruses. In comparison, as shown in FIGS. 26A-C and 27A-C, these four samples are discriminated from the normal database by using the method and apparatus of the present invention. Accordingly, the disease detection method and apparatus of the present invention provide earlier detection of infection, such as HIV infection, when it is yet to be detectable by conventional detection means.

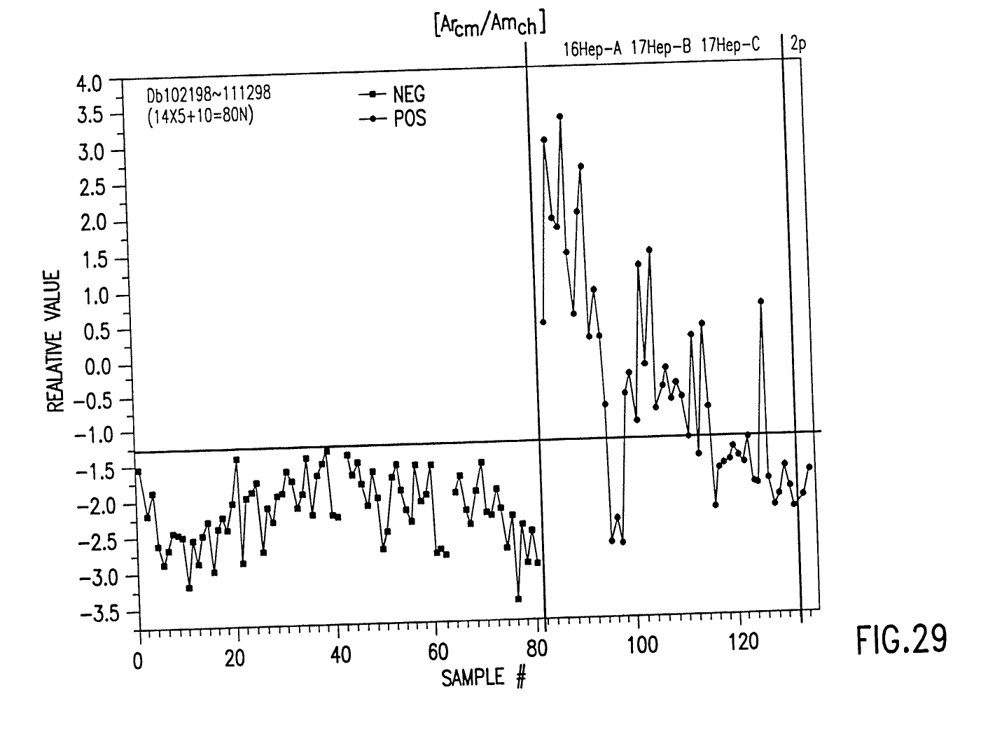

In accordance with the present invention, the disease detection method and apparatus of the present invention can be used to discriminate Hepatitis A, B, and C. Referring to FIG. 29, the x-axis is the sample number and the y-axis is (Am_{CM}-Am_{CH}), where Am_{CM} is the peak amplitude of the emission spectrum for the AGB-treated sample and Am_{CH} is the peak amplitude of the emission spectrum for the charcoal-treated samples. Sample Nos. 1-80 are samples from the normal database (i.e., Hepatitis negative samples). Sample Nos. 81-100 are samples known to be Hepatitis A positive. Sample Nos. 101-120 are samples known to be Hepatitis B positive. Sample Nos. 121-127 are samples known to be hepatitis C positive. As shown in FIG. 29, the parameter (Am_{CM}-Am_{CH}) is a discriminator between normal samples and hepatitis A or B positive samples. In addition, this parameter is also a discriminator between Hepatitis A samples and Hepatitis C samples.

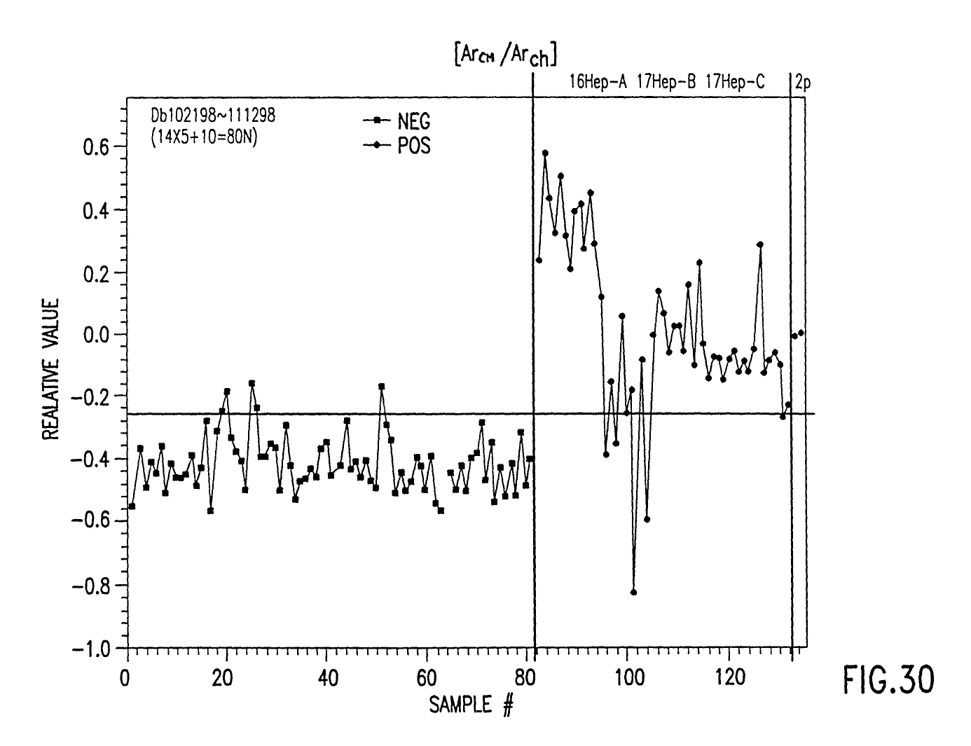

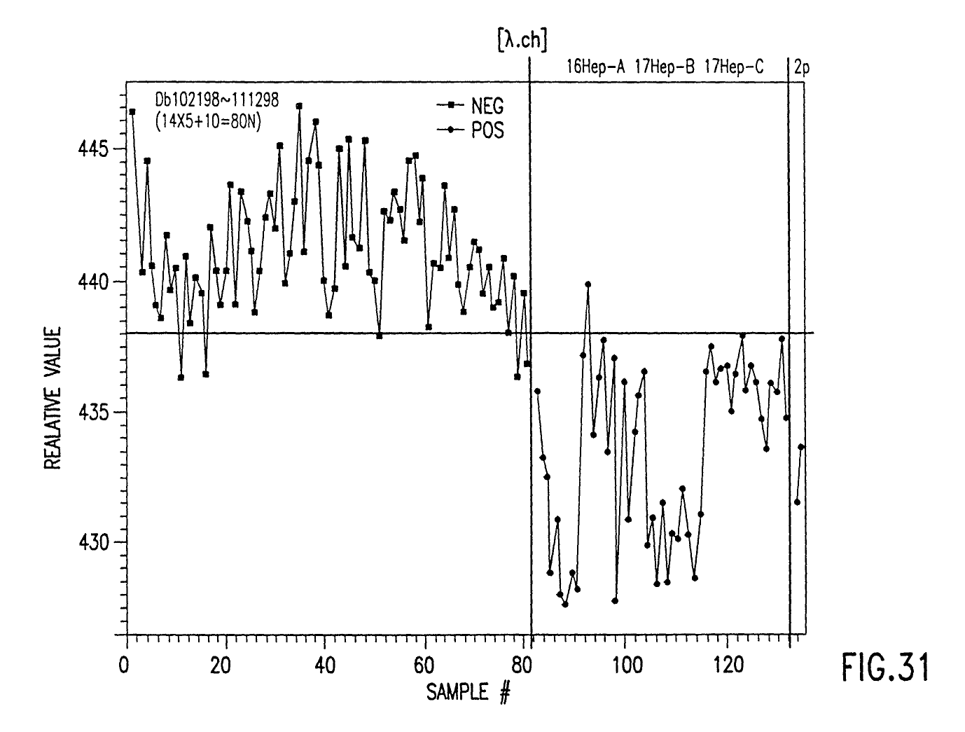

FIG. 30 shows the parameter (Ar_{CM}-Ar_{CH}), i.e., area ratio difference between AGB-treated and charcoal-treated samples, for the same set of samples of FIG. 29. It is shown that this parameter discriminates Hepatitis A and hepatitis C from normal samples.

FIG. 31 shows the peak wavelength, λ_{CH}, measurement of charcoal-treated samples, which indicates that this parameter discriminates Hepatitis A, B and C, respectively, from the normal samples.

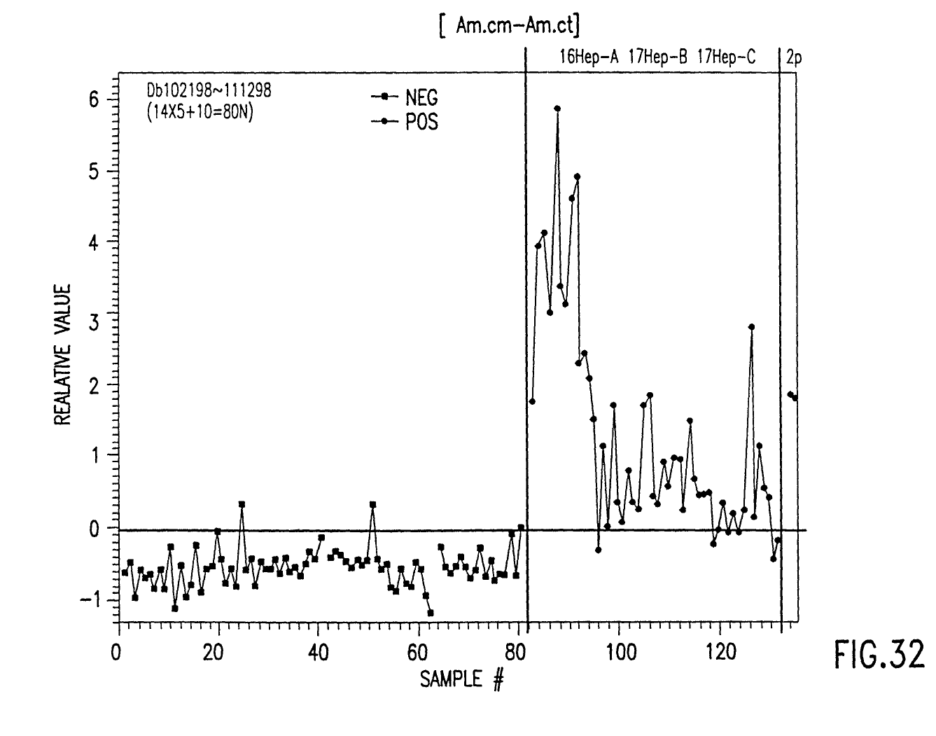

FIG. 32 shows the parameter (Am_{CM}-Am_{CT}), i.e., peak amplitude differences between CM-AGB treated samples and non-treated samples (control samples), which indicate that this parameter discriminates Hepatitis A, B and C, respectively, from the normal samples. Moreover, this parameter can also be used to differentiate Hepatitis A from Hepatitis C.

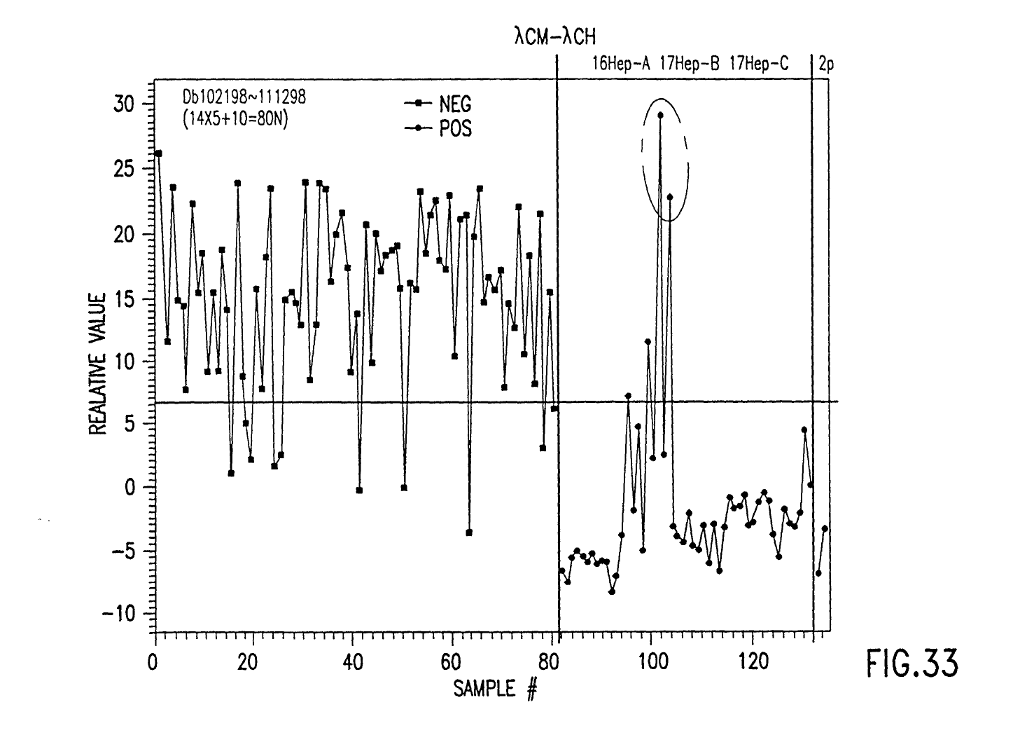

FIG. 33 shows the parameter (λ_{CM}-λ_{CM}), i.e., peak wavelength differences between CM-AGB treated samples and charcoal-treated samples, which shows that this parameter distinguishes Hepatitis A and C from normal samples.

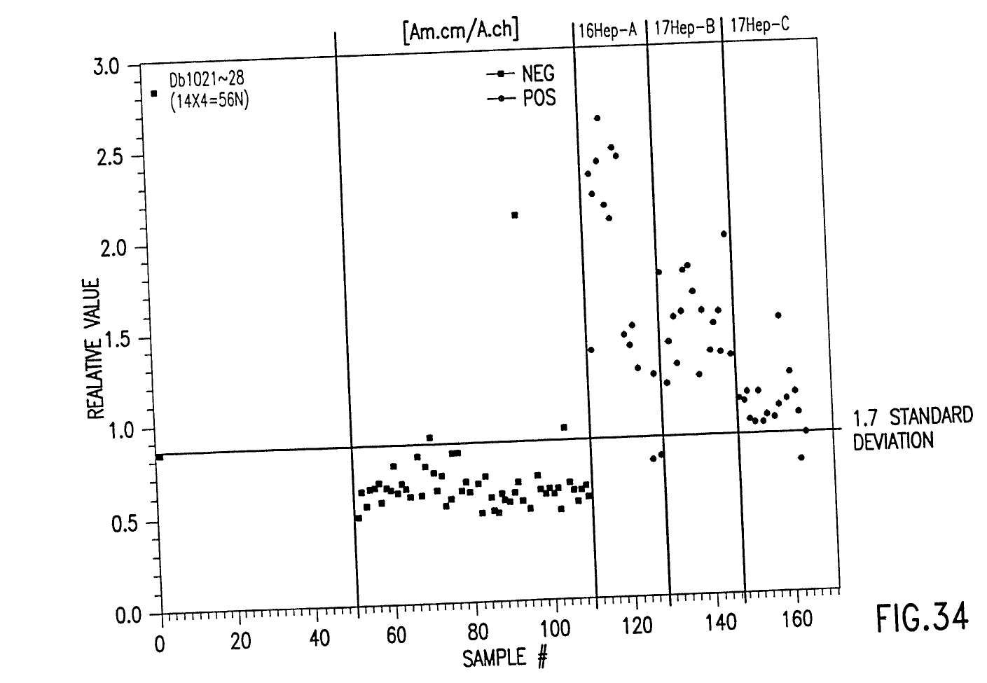

FIG. 34 shows the parameter (Am_{CM}/λ_{CH}), i.e., the amplitude of the CM-AGB treated samples divided by the peak wavelength of the charcoal-treated samples, which demonstrate that this parameter is a partial discriminator for Hepatitis A and C from normal samples, and is a good discriminator for Hepatitis B and normal samples.

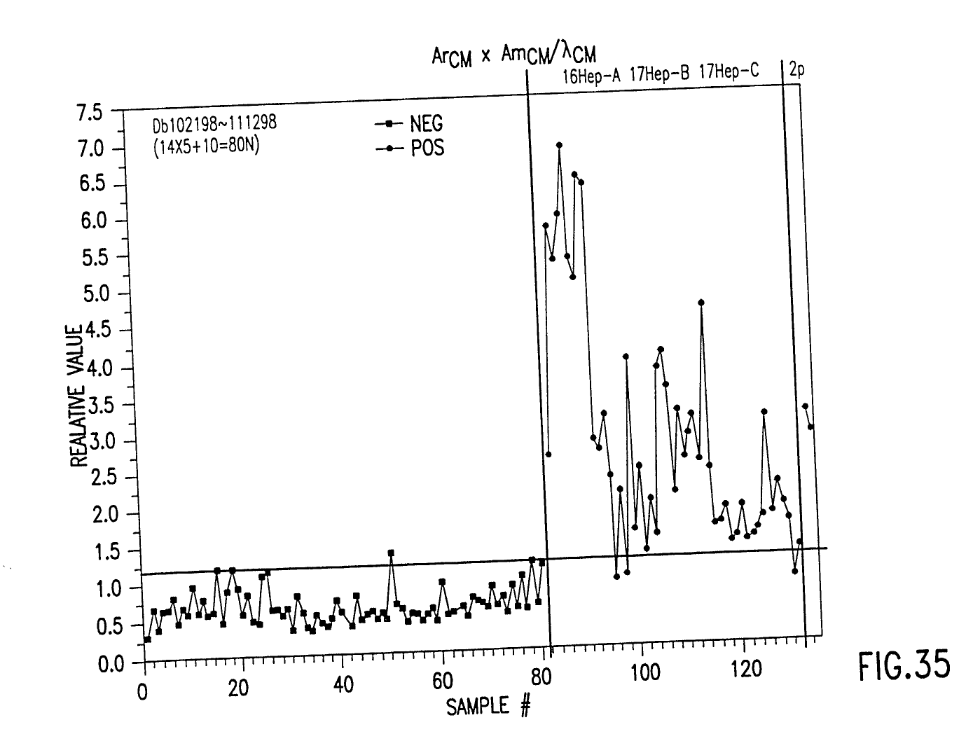

FIG. 35 shows a composite parameter (Ar_{CM}*Am_{CM}/λ_{CM}), i.e., the area ratio of the CM-AGB treated samples times the amplitude of such CM-AGB treated samples divided by the peak wavelength of such CM-AGB treated samples, which differentiates Hepatitis A, B, and C from the normal samples.

In all of the above samples, the excitation wavelength and the observed spectrum range are the same as that described above for the HIV detection. This demonstrates that the disease detection method and apparatus of the present invention is capable of being adapted for detection of a variety of viruses present in samples, such as HIV viruses, and Hepatitis A, B and C.

It will be apparent to one of ordinary skill in the art that the various other parameters may be adapted in accordance with the present invention, which may be used to discriminate a wide variety of diseases, such as Hepatitis A, B and C. The following examples provides additional parameters for detection of Hepatitis A, B and C.

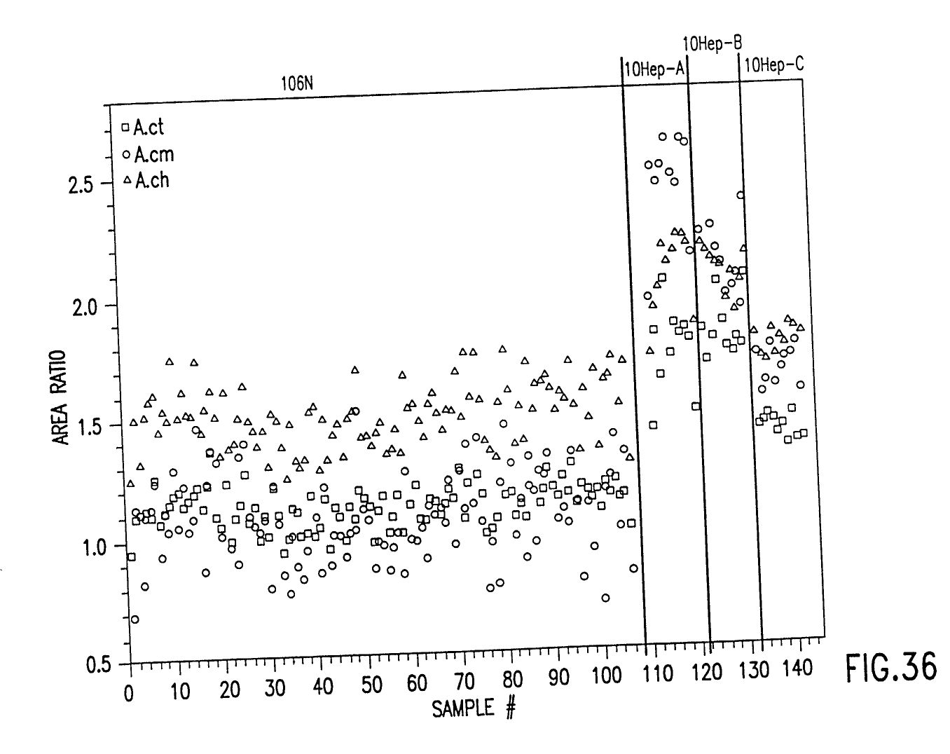

FIG. 36 shows the parameter of area ratios, Ar, for control samples, Ar_{CT}, (i.e., non-treated samples), represented as small squares; CM-AGB treated samples, Ar_{CM}, represented as small circles; and charcoal-treated samples, Ar_{CH}, represented as small triangles. It is shown that the parameter Ar_{CT} is a good discriminator for Hepatitis A, B and C, and the normal samples.

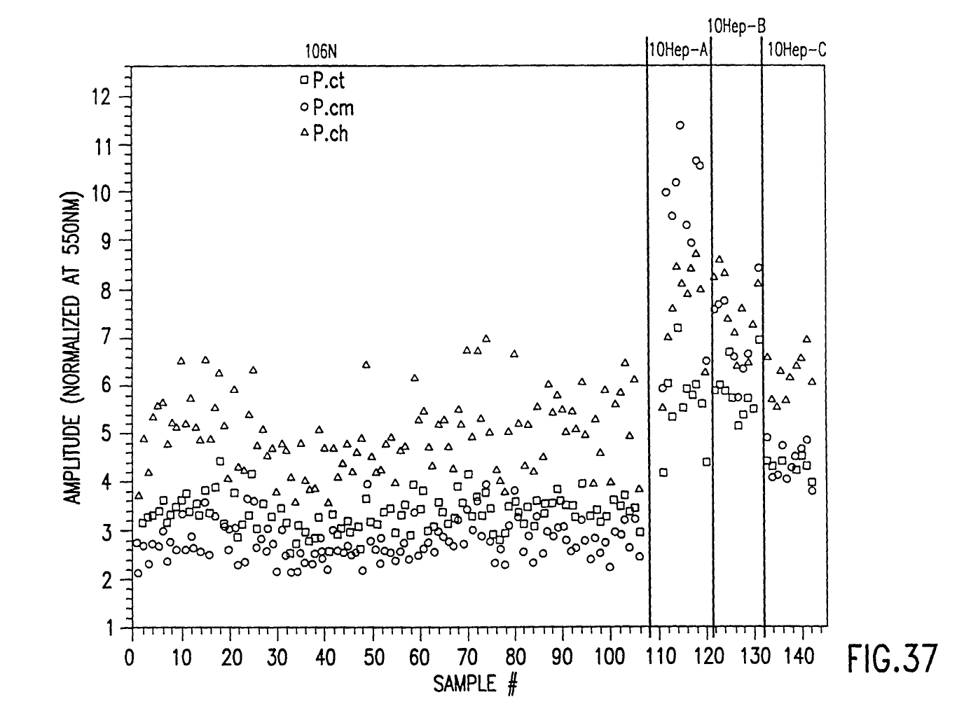

FIG. 37 shows the normalized peak amplitude, Am, for control samples, Am_{CT}, (i.e., non-treated samples), represented as small squares; CM-AGB treated samples, Am_{CM}, represented as small circles; and charcoal-treated samples, AM_{CH}, represented as small triangles.

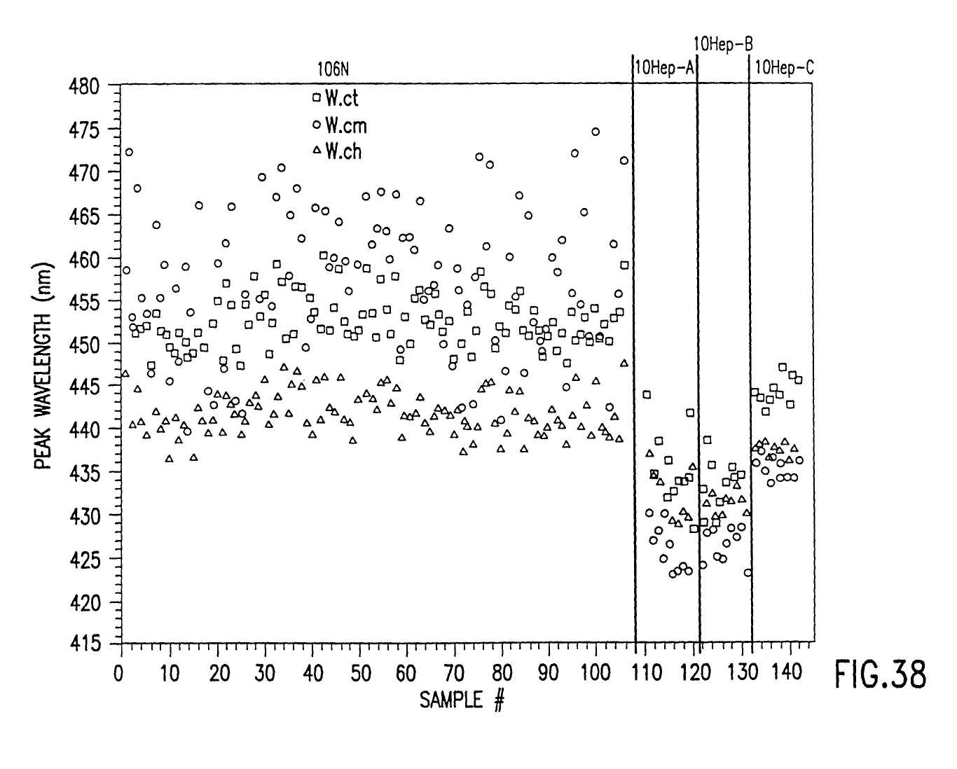

FIG. 38 shows the peak wavelength, λ_{P}, for control samples, λ_{CT}, (i.e., non-treated samples), represented as small squares; CM-AGB treated samples, λ_{CM}, represented as small circles; and charcoal-treated samples, λ_{CH}, represented as small triangles.

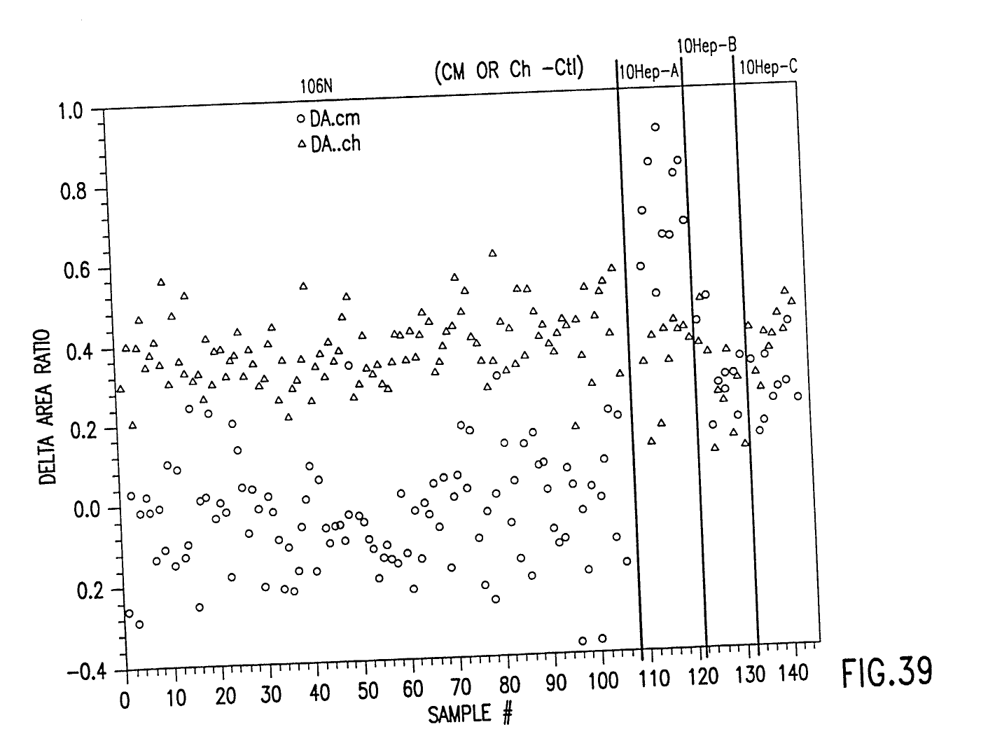

FIG. 39 shows the area ratio changes between CM-AGB treated samples and control samples, (Ar_{CM}-Ar_{CT}), represented as small circles; and area ratio changes between charcoal treated samples and control samples, (Ar_{CH}-Ar_{CT}), represented as small triangles.

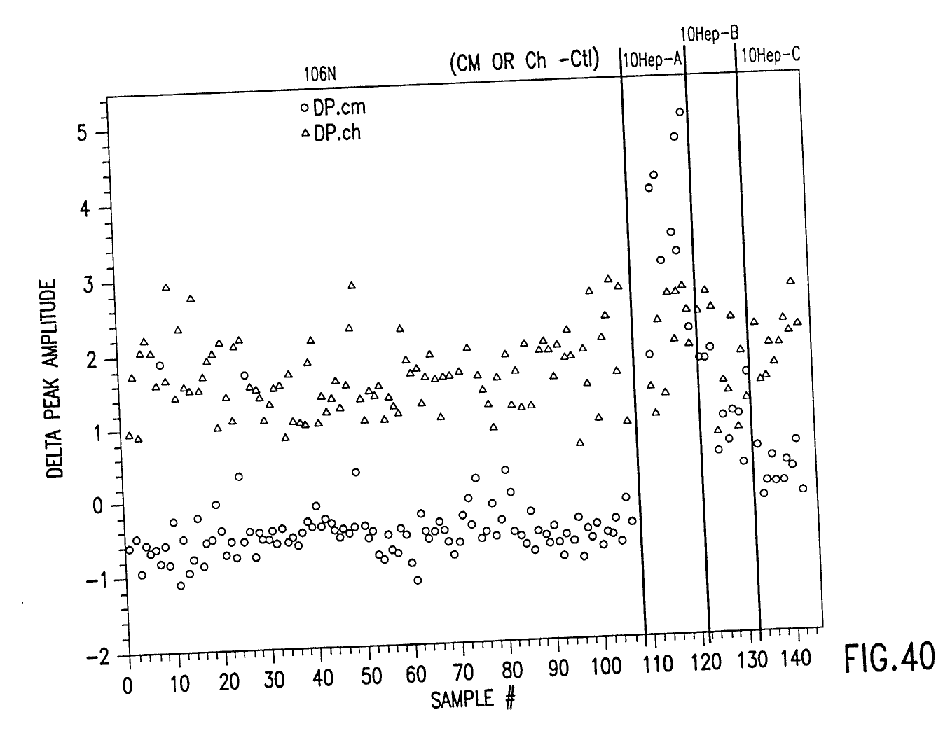

FIG. 40 shows the peak amplitude changes between CM-AGB treated samples and control samples, (Am_{CM}-Am_{CT}), represented as small circles; and peak amplitude changes between charcoal treated samples and control samples, (Am_{CH}-Am_{CT}), represented as small triangles.

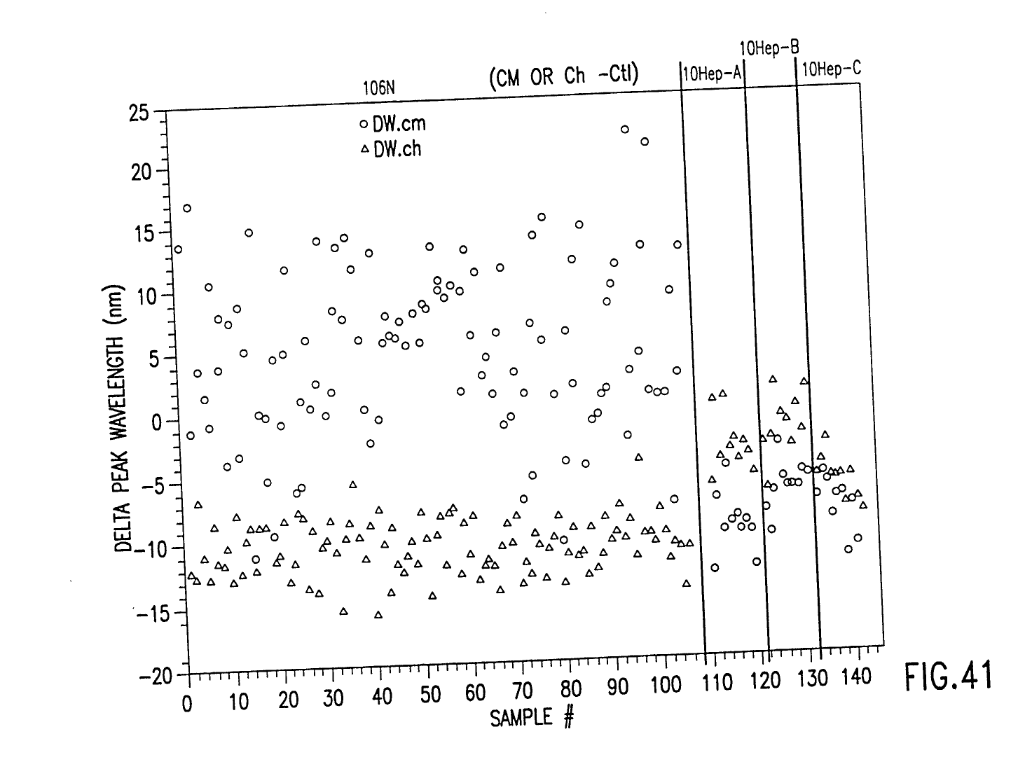

FIG. 41 shows the peak wavelength changes between CM-AGB treated samples and control samples, (λp_{CM}-λp_{CT}), represented as small circles; and peak wavelength changes between charcoal treated samples and control samples,(λp_{CH}-λp_{CT}), represented as small triangles.

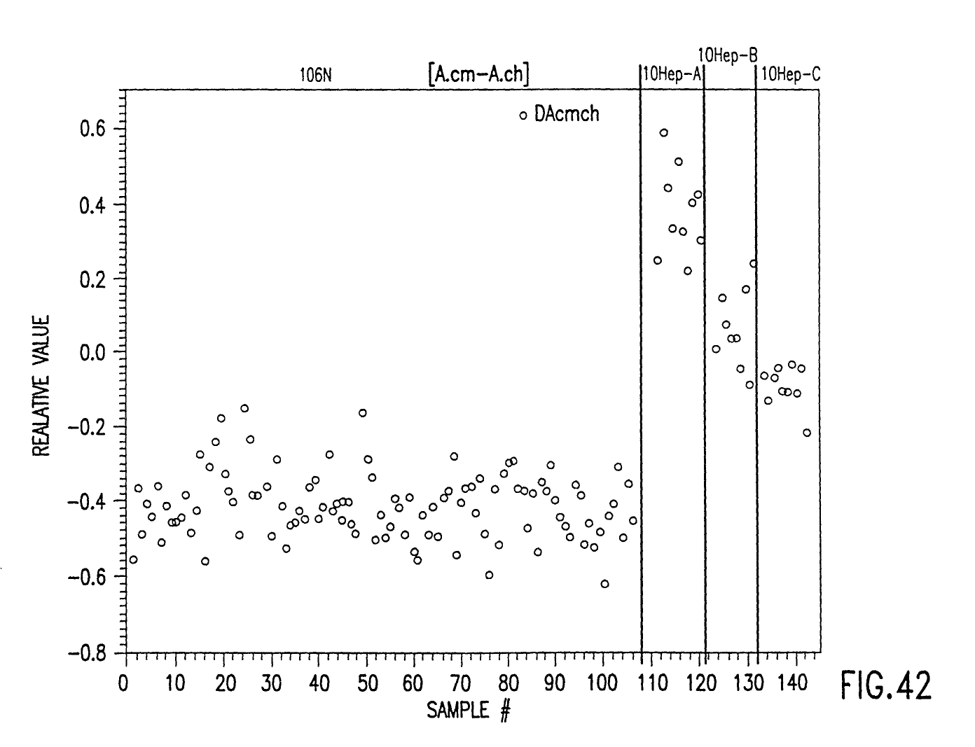

FIG. 42 shows the area ratio changes between CM-AGB treated samples and charcoal-treated samples, (Ar_{CM}-Ar_{CH}), represented as small circles. It clearly shows that this parameter is a good discriminator for normal samples and the samples containing Hepatitis A, B or C.

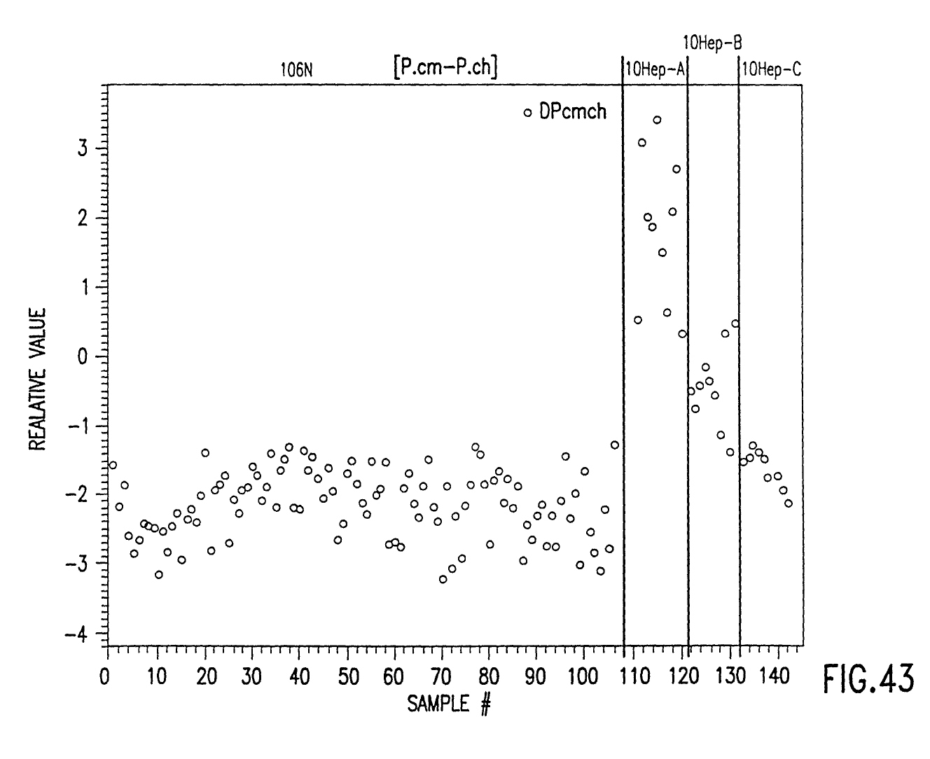

FIG. 43 shows the amplitude changes between CM-AGB treated samples and charcoal-treated samples, (Am_{CM}-Am_{CH}), represented as small circles.

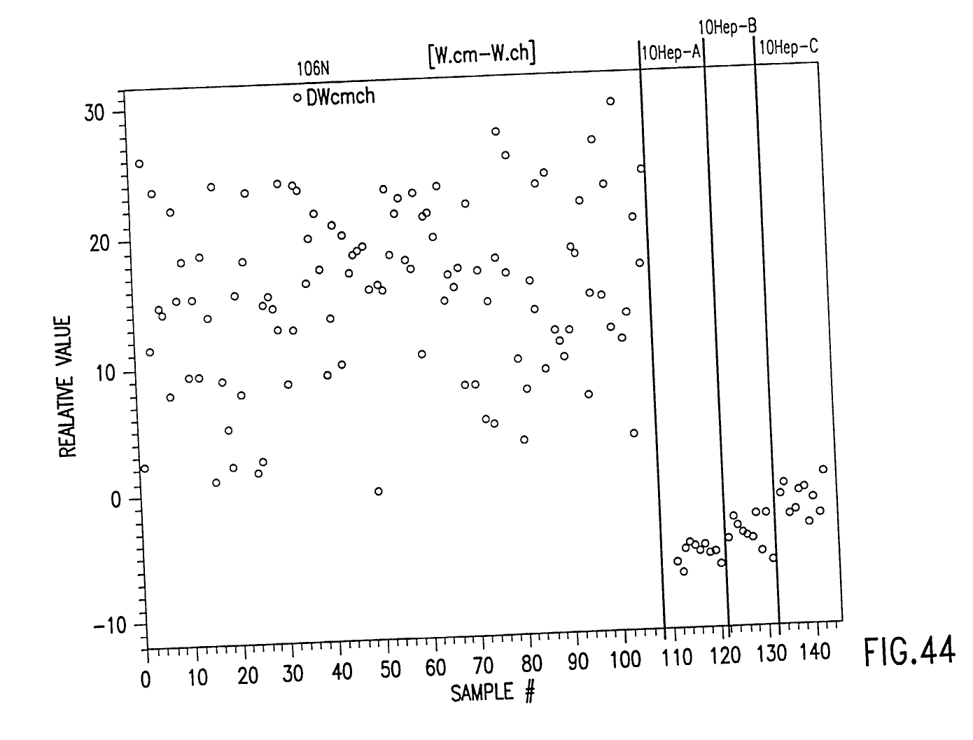

FIG. 44 shows the peak wavelength changes between CM-AGB treated samples and charcoal-treated samples, (λp_{CM}-λp_{CH}), represented as small circles. This parameter clearly discriminates the normal samples from those containing Hepatitis A, B or C.

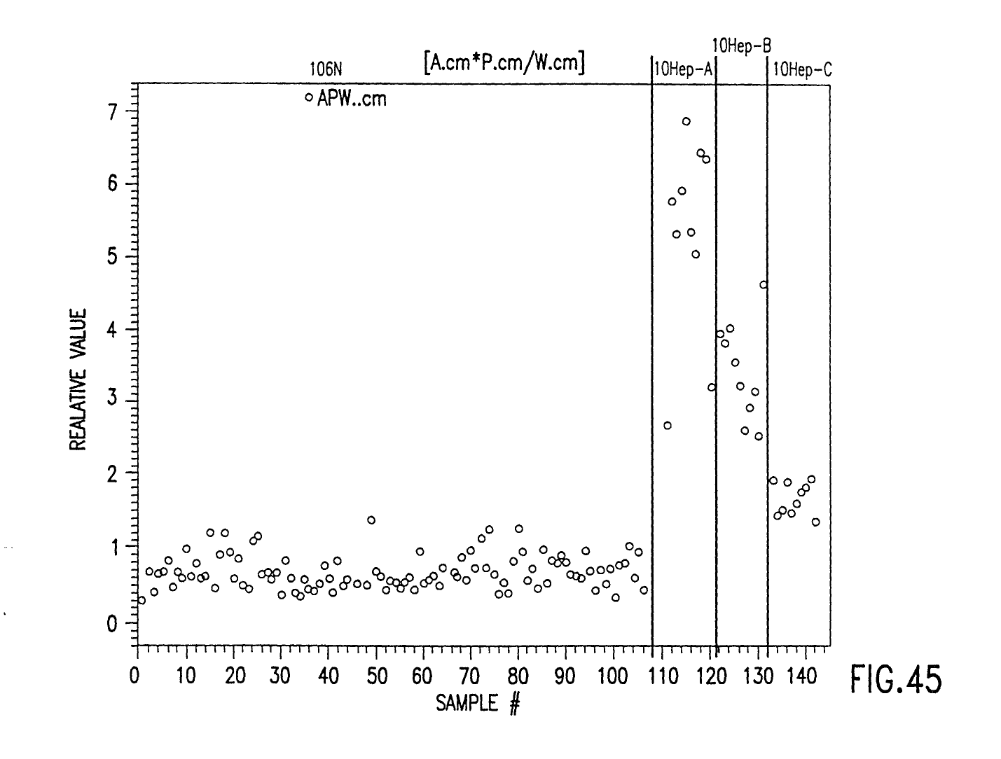

FIG. 45 shows a composite parameter, area ratio times the amplitude divided by the peak wavelength, Ar_{CM}*Am_{CM/λp}_{CM}, for samples treated with CM-AGB, represented as small circles. This parameter at least differentiates the normal samples from Hepatitis A or B positive samples.

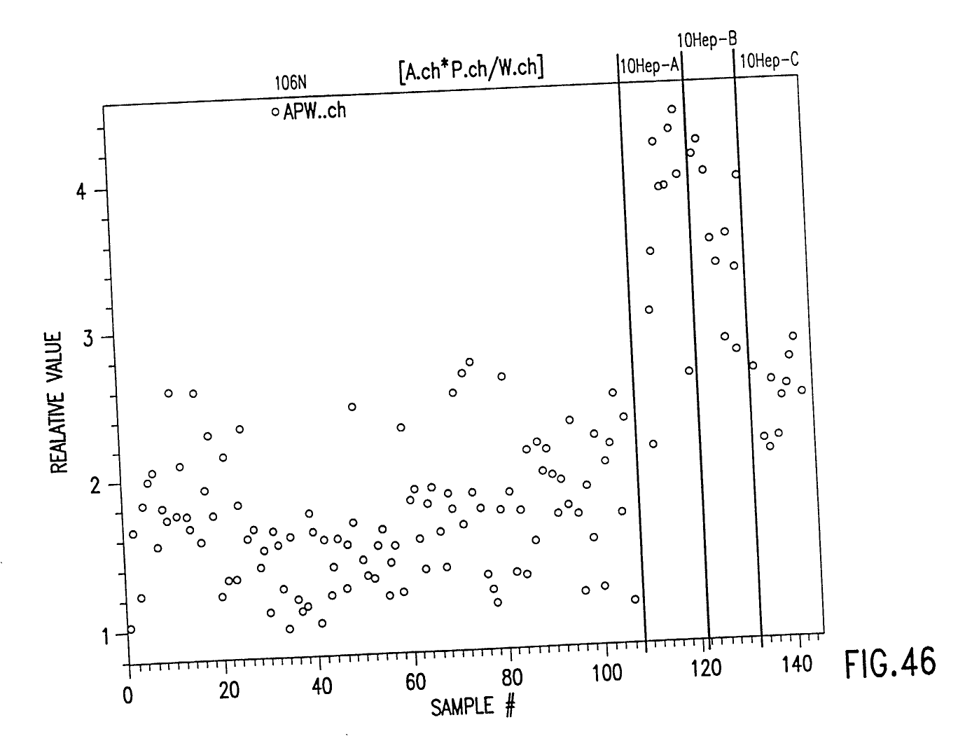

FIG. 46 shows another composite parameter, area ratio times the amplitude divided by the peak wavelength, Ar_{CH}*Am_{CH}/λp_{CH}, for samples treated with charcoal, represented as small circles.

The preferred embodiment described above is not intended to limit the applicability of the present invention. Rather, a person skilled in the art would appreciate that various modifications of the arrangements and types of components used in the detection system not mentioned in the specifications of the present invention would fall within the scope and spirit of the claimed invention.

What is claimed is:

1. A method for detecting presence of a diseased state, comprising:
a. obtaining a specimen;
b. preparing a sample from the specimen;
c. irradiating the sample;
d. obtaining at least one of absorption and emission spectra from the irradiated sample; and
e. analyzing the at least one of the absorption and emission spectra to determine the presence of the diseased state.

2. A method for detecting presence of an infection, comprising:
a. obtaining a specimen;
b. preparing a sample from the specimen;
c. irradiating the sample;
d. obtaining at least one of absorption and emission spectra from the irradiated sample; and
e. analyzing the at least one of the absorption and emission spectra to determine the presence of the infection.

3. A method for detecting presence of an infection, comprising:
a. obtaining a specimen;
b. preparing a sample from the specimen;
c. irradiating the sample;
d. obtaining at least one of absorption and emission spectra from the irradiated sample; and
e. analyzing the at least one of absorption and emission spectra to determine variation of the at least one of the absorption and emission spectra from a spectrum characteristic of a non-infected state to detect the presence of the infection.

4. A method comprising:
a. preparing a sample from an organism;
b. irradiating the sample;
c. obtaining at least one of absorption and emission spectra from the irradiated sample; and
d. analyzing the at least one of the absorption and emission spectra to determine the state of health of the organism.

5. An apparatus for detecting presence of a diseased state in a sample, comprising:
a. radiation source; and
b. analyzer operatively associated with the sample for measuring at least one of absorption and emission spectra from the sample to detect the presence of the diseased state in the sample.

6. The apparatus of claim 5 wherein the analyzer comprises a comparator to compare the at least one absorption and emission spectra with a signal characteristic of a non-diseased state.

7. The apparatus of claim 6 further comprising a memory unit coupled with the comparator and containing a plurality of pre-determined signals characteristic of non-diseased states.

8. The apparatus of claim 5 further comprising a sample holder disposed relative to the radiation source so that at least a portion of the sample can be irradiated.

9. The apparatus of claim 8 wherein the sample holder is a flow-through cell.

10. The apparatus of claim 5 wherein the radiation source emits electromagnetic radiation between about 270 and 400 nanometers.

11. The apparatus of claim 10 wherein the radiation source is a laser.

12. The apparatus of claim 10 wherein the radiation source comprises a filtered light source.

13. The apparatus of claim 5 wherein the analyzer comprises a spectrometer.

14. The apparatus of claim 13 wherein the spectrometer is a time-resolved spectrometer.

15. The apparatus of claim 13 wherein the spectrometer includes a polarizer.

16. The apparatus of claim 5 wherein the sample is one of plasma, serum, blood articles, and blood.

17. The apparatus of claim 5 wherein the sample is treated with an absorbent.

18. The apparatus of claim 17 wherein the absorbent is C-M Affi Gel Blue.

19. The apparatus of claim 17 wherein the absorbent is activated charcoal.

20. The apparatus of claim 5 wherein the analyzer measures at least one of a wavelength of maximum amplitude, a maximum intensity of the wavelength of maximum amplitude, and a predetermined area ratio of the at least one of absorption and emission spectra from the sample.

21. The apparatus of claim 5 wherein the diseased state is HIV.

22. The apparatus of claim 5 wherein the diseased state is hepatitis.

23. The apparatus of claim 5 wherein the analyzer measures the emission spectrum.

24. An apparatus for detecting presence of a diseased state, comprising:
a. means for irradiating the sample;
b. means for obtaining at least one of absorption and emission spectra from the irradiated sample; and
c. means for analyzing the at least one of the absorption and emission spectra to determine the presence of the diseased state.
